(19) United States
(12) Patent Application Publication
(10) Pub. No.: US 2021/0275369 A1
(43) Pub. Date: Sep. 9, 2021

(54) WALKING SUPPORT SYSTEM FOR COMBINED TRAINING

(71) Applicant: JTEKT CORPORATION, Osaka-shi (JP)

(72) Inventors: Yoshiyuki SHIBATA, Toyota-shi (JP); Shinji TAKEUCHI, Okazaki-shi (JP); Yuki MATSUOKA, Kariya-shi (JP); Manabu KANAYA, Nara-shi (JP)

(73) Assignee: JTEKT CORPORATION, Osaka-shi (JP)

(21) Appl. No.: 17/188,203

(22) Filed: Mar. 1, 2021

(30) Foreign Application Priority Data
Mar. 6, 2020 (JP) 2020-038511

(51) Int. Cl. A61G 5/02; G16H 20/60; G16H 10/20; G16H 40/63

(52) U.S. Cl. CPC A61G 5/022; G16H 40/63; G16H 10/20; G16H 20/60

(57) ABSTRACT

There is provided a walking support system for a combined training including: a walking assist device configured to move in accordance with an operation of a user to assist a walking of the user; a voice output unit configured to output a voice; and a task load unit configured to perform a voice-output for a task to the user via the voice output unit when the walking assist device is used by the user during the walking, the task being answerable by the voice.

### CROSS-REFERENCES TO RELATED APPLICATIONS

This application is based on and claims priority under 35 USC 119 from Japanese Patent Application No. 2020-038511 filed on Mar. 6, 2020, the contents of which are incorporated herein by reference.

### TECHNICAL FIELD

The present disclosure relates to a walking support system for combined training.

### BACKGROUND ART

Various techniques have been proposed to support walking of an elderly person or the like. For example, according to a walking assist device described in JP2016-154718A, an operation force detection device that detects an operation force acting on a grip is provided in the grip which is mounted outside a handle. The operation force detection device includes a sliding pipe that is slidable in an axial direction in accordance with gripping, and a fixed pipe arranged inside the sliding pipe. One end of the fixed pipe is fixed to a main frame of the walking assist device.

The fixed pipe includes a pair of pressure sensitive resistance sensors which are fixed to face each other in the axial direction relative to the fixed pipe so as to detect an operation force acting in the axial direction of the sliding pipe, and are spaced apart from each other by a predetermined distance. The sliding pipe includes a pressing member which is fixed to the sliding pipe, arranged between the pair of pressure sensitive resistance sensors, and configured to transmit a force acting in the axial direction of the sliding pipe to the pair of pressure sensitive resistance sensors. The pressing member includes a pair of sensor abutment portions which protrude from two axial direction end surfaces thereof.

When an operation force of a user acts on the sliding pipe from the grip, one of the pair of sensor abutment portions, which protrude from the two axial direction end portions of the pressing member coupled to the sliding pipe, presses the opposite pressure sensitive resistance sensor. The pressure sensitive resistance sensor pressed by the sensor abutment portion outputs a detection signal to a control unit of the walking assist device. The control unit is configured to detect the operation force acting on the handle based on the input detection signal, and to drive and control the walking assist device.

However, according to the walking assist device described in JP2016-154718A, although the elderly person or the like can walk comfortably by using the walking assist device, walking ability thereof gradually decreases. Therefore, there may be a high possibility of falling during walking without using the walking assist device. Meanwhile, a method of performing dual task (double task) training is known as a method of fall prevention. The dual task training refers to training that activates a brain by increasing blood flow in a frontal lobe (which controls movement and thinking) of the brain by simultaneously performing exercises such as walking while performing tasks such as calculation and speaking words that need to be recalled.

### SUMMARY OF INVENTION

The present disclosure provides a walking support system for combined training capable of performing dual task training and more effective walking training when a user uses a walking assist device to walk.

According to a first aspect of the present disclosure, a walking support system for a combined training includes: a walking assist device configured to move in accordance with an operation of a user to assist a walking of the user; a voice output unit configured to output a voice; and a task load unit configured to perform a voice-output for a task to the user via the voice output unit when the walking assist device is used by the user during the walking, the task being answerable by the voice.

According to a second aspect of the present disclosure, the walking support system of the first aspect further includes: a voice input unit configured to receive a voice answer to the task from the user; an answer analysis unit configured to analyze the voice answer received via the voice input unit; and a notification unit configured to notify an analysis information of the voice answer provided by the answer analysis unit.

According to a third aspect of the present disclosure, the walking support system of the second aspect further includes: an analysis information collection unit configured to collect the analysis information of the voice answer provided by the answer analysis unit; an answer evaluation unit configured to evaluate an execution degree of the task based on the analysis information collected by the analysis information collection unit; and a teaching unit configured to teach an evaluation information related to the execution degree of the task evaluated by the answer evaluation unit.

According to a fourth aspect of the present disclosure, the walking support system of the third aspect further includes a reward granting unit configured to determine and grant a reward to the user in accordance with the execution degree of the task evaluated by the answer evaluation unit.

According to a fifth aspect of the present disclosure, in the walking support system of any one of the first aspect to the fourth aspect, the walking assist device includes a device communication device. The walking support system further includes a mobile terminal including a terminal communication device which is communicable with the device communication device. The mobile terminal includes the voice output unit and the task load unit.

According to a sixth aspect of the present disclosure, in the walking support system of the fifth aspect, the walking assist device includes an attachment member to which the mobile terminal is detachably attached.

According to a seventh aspect of the present disclosure, the walking support system of any one of the second aspect to the sixth aspect further includes: an elapsed time measurement unit configured to measure an elapsed time elapsed after the voice-output the task is performed via the task load unit; and a time determination unit configured to determine whether the elapsed time reaches a predetermined first elapsed time in a state where the voice answer to the task is not received via the voice input unit. When the time determination unit determines that the elapsed time reaches the predetermined first elapsed time, the task load unit performs again the voice-output of the task via the voice output unit.

According to an eighth aspect of the present disclosure, the walking support system of any one of the second aspect to the sixth aspect further includes: an elapsed time measurement unit configured to measure an elapsed time elapsed after the voice-output of the task is performed via the task load unit; and a time determination unit configured to determine whether the elapsed time reaches a predetermined first elapsed time in a state where the voice answer to the task is not received via the voice input unit. When the time determination unit determines that the elapsed time reaches the predetermined first elapsed time, the task load unit changes the task to another task and performs the voice-output of the another task via the voice output unit.

According to a ninth aspect of the present disclosure, in the walking support system of the third aspect, the walking assist device includes a device communication device. The walking support system further includes: a mobile terminal including a terminal communication device which is communicable with the device communication device; and a server including a server communication device which is communicable with the terminal communication device. The mobile terminal includes: the voice output unit, the task load unit, the voice input unit, the answer analysis unit, the notification unit, the analysis information collection unit, and the teaching unit; a terminal transmission unit configured to transmit the analysis information collected by the analysis information collection unit to the server via the terminal communication device; and a terminal reception unit configured to receive the evaluation information related to the execution degree of the task from the server via the terminal communication device. The server includes: a server reception unit configured to receive the analysis information collected by the analysis information collection unit via the server communication device; the answer evaluation unit configured to evaluate the execution degree of the task based on the analysis information which is received by the server reception unit and collected by the analysis information collection unit; and a server transmission unit configured to transmit the evaluation information related to the execution degree of the task evaluated by the answer evaluation unit to the mobile terminal via the server communication device.

According to a tenth aspect of the present disclosure, in the walking support system of the third aspect, the walking assist device includes a device communication device. The walking support system further includes a server including a server communication device which is communicable with the device communication device. The walking assist device includes: the voice output unit, the task load unit, the voice input unit, the answer analysis unit, the notification unit, the analysis information collection unit, and the teaching unit; a device transmission unit configured to transmit the analysis information collected by the analysis information collection unit to the server via the device communication device; and a device reception unit configured to receive the evaluation information related to the execution degree of the task from the server via the device communication device. The server includes: a server reception unit configured to receive the analysis information collected by the analysis information collection unit via the server communication device; the answer evaluation unit configured to evaluate the execution degree of the task based on the analysis information which is received by the server reception unit and collected by the analysis information collection unit; and a server transmission unit configured to transmit the evaluation information related to the execution degree of the task evaluated by the answer evaluation unit to the walking assist device via the server communication device.

According to an eleventh aspect of the present disclosure, in the walking support system of any one of the first aspect to the tenth aspect, the task includes at least one of a calculation question, a four-basic-math-operation question, a quiz question, and a question of a menu of a meal eaten by the user.

According to the first aspect, when the user operates the walking assist device and walks, the task that can be answered by voice is voice-output to the user. Accordingly, the user can answer by voice in consideration of the answer to the task each time the task is voice-output while walking training using the walking assist device is performed. As a result, the user can perform the dual task training in which the walking training is performed at the same time while answering by voice in consideration of the answer to the task, and thus the walking training can be performed more effectively.

According to the second aspect, the analysis information of the voice answer to the task from the user can be notified. For example, analysis information indicating whether the voice answer from the user is correct can be notified. As a result, interest of the user in providing the voice answer to the task during the walking training can be attracted, and thus the dual task training can be effectively performed.

According to the third aspect, the analysis information of the voice answer to the task from the user is collected. Then, based on the collected analysis information, the execution degree of the task executed by the user is evaluated, and the evaluation information (for example, daily correct answer rate, number of correct answers, and changes in answering time) related to the execution degree of the task is taught to the user or the like. As a result, the interest of the user in providing the voice answer to the task during the walking training can be effectively attracted by the taught evaluation information, and motivation for the user to continuously perform the dual task training can be increased.

According to the fourth aspect, since the reward is granted to the user in accordance with the execution degree of the task, the interest of the user in providing the voice answer to the task can be more effectively attracted, and the motivation for the user to continuously perform the dual task training can be increased.

According to the fifth aspect, the mobile terminal (for example, a smartphone or a tablet) can voice-output the task that can be answered by voice to the user. Accordingly, with a simple configuration, the user can answer by voice in consideration of the answer to the task each time the task is voice-output by the mobile terminal while the walking training using the walking assist device is performed. As a result, the user can perform the dual task training in which the walking training is performed at the same time while answering by voice in consideration of the answer to the task, and thus the walking training can be performed more effectively.

According to the sixth aspect, the mobile terminal is detachably attached to the attachment member provided on the walking assist device. As a result, a communication distance between the device communication device of the walking assist device and the terminal communication device of the mobile terminal can be shortened, and thus communication can be reliably performed. Moreover, the user can easily operate the mobile terminal while using the walking assist device to perform the walking training. Moreover, when the walking training is completed, the user can remove the mobile terminal from the attachment member and carry the mobile terminal.

According to the seventh aspect, when the voice answer to the task is not received from the user until the elapsed time from the time when the task is voice-output reaches the predetermined first elapsed time, the task is voice-output again. As a result, even when the user fails to hear the task while performing the walking training, the user can hear the task again, and can answer the task by voice in consideration of the answer to the task. As a result, the user can perform the dual task training in which the walking training is performed at the same time while answering by voice in consideration of the answer to the task, and thus the walking training can be performed more effectively.

According to the eighth aspect, when the voice answer to the task is not received from the user until the elapsed time from the time when the task is voice-output reaches the predetermined first elapsed time, the task is changed and the changed task is voice-output. As a result, even when the user does not know the answer to the task while performing the walking training, the user can hear the changed task again, and can answer the task by voice in consideration of the answer to the task. As a result, the interest of the user in providing the voice answer to the task during the walking training can be attracted, and thus the dual task training can be effectively performed.

According to the ninth aspect, the mobile terminal device transmits the analysis information collected by the analysis information collection unit to the server. Meanwhile, the server evaluates the execution degree of the task executed by the user based on the received analysis information collected by the analysis information collection unit. Then the server transmits the evaluation information related to the execution degree of the task to the mobile terminal. Thereafter, the mobile terminal receives the evaluation information related to the execution degree of the task transmitted from the server, and teaches the evaluation information to the user.

As a result, since the server executes processing of the answer evaluation unit that evaluates the execution degree of the task executed by the user based on the analysis information collected by the analysis information collection unit, a processing load of the mobile terminal can be reduced. Moreover, it is possible to transmit the evaluation information related to the execution degree of the task from the server to not only the mobile terminal but also other processing devices.

According to the tenth aspect, the walking assist device transmits the analysis information collected by the analysis information collection unit to the server. Meanwhile, the server evaluates the execution degree of the task executed by the user based on the received analysis information collected by the analysis information collection unit. Then the server transmits the evaluation information related to the execution degree of the task to the walking assist device. Thereafter, the walking assist device receives the evaluation information related to the execution degree of the task transmitted from the server, and teaches the evaluation information to the user.

As a result, since the server executes the processing of the answer evaluation unit that evaluates the execution degree of the task executed by the user based on the analysis information collected by the analysis information collection unit, a processing load of the walking assist device can be reduced. Moreover, it is possible to transmit the evaluation information related to the execution degree of the task from the server to not only the walking assist device but also other processing devices.

According to the eleventh aspect, the voice-output task includes at least one of the calculation question, the four-basic-math-operation question, the quiz question, or the question of the menu of the meal eaten by the user. As a result, a question that the user needs to think about or recall can be given as the task. As a result, the user can effectively perform the dual task training in which the walking training is performed at the same time while answering by voice in consideration of the answer to the task, and thus the walking training can be performed more effectively.

### BRIEF DESCRIPTION OF DRAWINGS

FIG. 1 is a perspective view showing a schematic configuration of a walking support system for combined training according to a first embodiment.

FIG. 2 is a cross-sectional view taken along line II-II of FIG. 1 which shows an example of structures of a grip handle and an operation force direction detection device arranged on a left handle frame.

FIG. 3 is a block diagram showing a control configuration of a walking assist device.

FIG. 4 shows an example of a drive table that stores correspondence between operation force directions of left and right grip handles and a traveling-or-turning mode.

FIG. 5 is a block diagram showing a schematic configuration of a smartphone.

FIG. 6 is a block diagram showing a configuration of a control device shown in FIG. 5.

FIG. 7 shows an example of a display screen of the smartphone.

FIG. 8 is a main flowchart showing an example of a “walking support process” executed by a control device of the walking assist device.

FIG. 9 is a sub-flowchart showing an example of a sub-process of a “travel start determination process” shown in FIG. 8.

FIG. 10 is a sub-flowchart showing an example of a sub-process of a “travel control process” shown in FIG. 8.

FIG. 11 is a sub-flowchart showing an example of a sub-process of a “battery process” shown in FIG. 8.

FIG. 12 is a main flowchart showing an example of a “first walking training process” executed by the control device of the smartphone.

FIG. 13 is a sub-flowchart showing an example of a sub-process of a “mode selection process” shown in FIG. 12.

FIG. 14 is a sub-flowchart showing an example of a sub-process of a “start or stop process” shown in FIG. 12.

FIG. 15 is a sub-flowchart showing an example of a sub-process of a “dual task process” shown in FIG. 12.

FIG. 16 is a sub-flowchart showing an example of a sub-process of an “evaluation and teaching process” shown in FIG. 12.

FIG. 17 shows an example of a screen of the smartphone where an evaluation result is displayed.

FIG. 18 is a block diagram showing a schematic configuration of a walking support system for combined training according to a second embodiment.

FIG. 19 is a block diagram showing a control configuration of a server.

FIG. 20 is a main flowchart showing an example of a “second walking training process” executed by a control device of a smartphone shown in FIG. 18.

FIG. 21 is a sub-flowchart showing an example of a sub-process of a “second evaluation and teaching process” shown in FIG. 20.

FIG. 22 is a flowchart showing an example of an “evaluation result creation process” executed by a control device of the server shown in FIG. 18.

FIG. 23 is a perspective view showing a schematic configuration of a walking support system for combined training according to a third embodiment.

FIG. 24 shows an example of a display screen of a display device.

FIG. 25 is a block diagram showing a control configuration of a walking assist device shown in FIG. 23.

FIG. 26 is a block diagram showing a configuration of a control device shown in FIG. 25.

FIG. 27 is a main flowchart showing an example of a “third walking training process” executed by the control device of the walking assist device shown in FIG. 23.

FIG. 28 is a sub-flowchart showing an example of a sub-process of a “second start or stop process” shown in FIG. 27.

FIG. 29 is a sub-flowchart showing an example of a sub-process of a “second battery process” shown in FIG. 27.

FIG. 30 is a sub-flowchart showing an example of a sub-process of a “second dual task process” shown in FIG. 27.

FIG. 31 is a sub-flowchart showing an example of a sub-process of a “third evaluation and teaching process” shown in FIG. 27.

FIG. 32 shows an example of a screen of the display device where an evaluation result is displayed.

FIG. 33 is a block diagram showing a schematic configuration of a walking support system for combined training according to a fourth embodiment.

FIG. 34 is a main flowchart showing an example of a “fourth walking training process” executed by a control device of a walking assist device shown in FIG. 33.

FIG. 35 is a sub-flowchart showing an example of a sub-process of a “fourth evaluation and teaching process” shown in FIG. 34.

FIG. 36 is a flowchart showing an example of a “second evaluation result creation process” executed by a control device of a server shown in FIG. 33.

### DESCRIPTION OF EMBODIMENTS

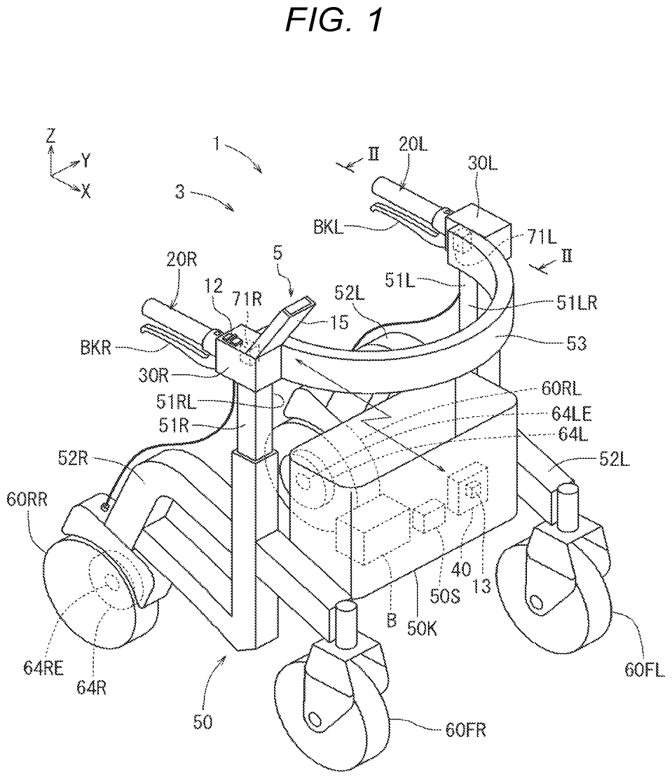

Hereinafter, a detailed description will be given with reference to the drawings based on first to fourth embodiments embodying a walking support system for combined training according to the present disclosure. First, a walking support system for combined training 1 according to the first embodiment will be described with reference to FIGS. 1 to 17. When an X-axis, a Y-axis, and a Z-axis are shown in the drawings, the respective axes are orthogonal to each other. An X-axis direction indicates a forward direction as viewed from a walking assist device 3, a Y-axis direction indicates a leftward direction as viewed from the walking assist device 3, and a Z-axis direction indicates an upward direction as viewed from the walking assist device 3.

### First Embodiment

As shown in FIG. 1, the walking support system for combined training 1 includes the walking assist device 3 and a smartphone 5 (mobile terminal). A tablet PC or the like may be used instead of the smartphone 5.

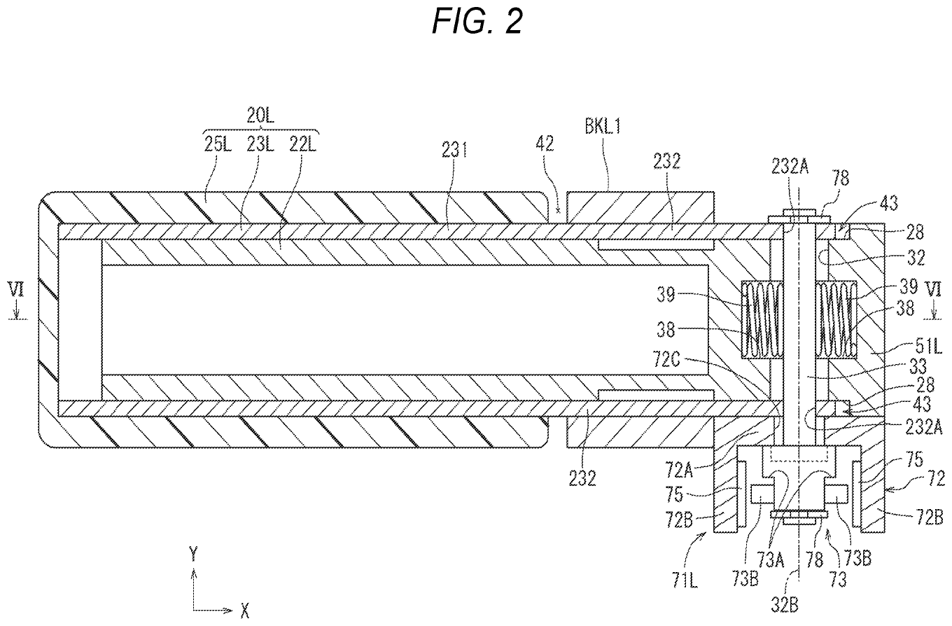

Next, a schematic configuration of the walking assist device 3 will be described with reference to FIGS. 1 and 2. As shown in FIG. 1, the walking assist device 3 includes a main frame 50, front wheels 60FL and 60FR, rear wheels 60RL and 60RR, travel drive devices 64L and 64R, a battery B, a control device 40, grip handles 20L and 20R, brake levers BKL and BKR, handle covers 30L and 30R, a bag 50K and the like.

The main frame 50 includes handle frames 51L and 51R, which extend in an up-down direction and support the grip handles 20L and 20R, wheel frames 52L and 52R, which extend in a main frame front-rear direction (front-rear direction relative to the main frame 50) and support the wheels, and the like. The wheel frame 52L is fixed below the handle frame 51L, and the wheel frame 52R is fixed below the handle frame 51R.

An elastically deformable coupling body 53 is provided on upper sides of the handle frame 51L and the handle frame 51R. A user enters between the wheel frame 52L and the wheel frame 52R from an opened side (rear side) of the main frame 50, grips the grip handle 20L and the grip handle 20R with left and right hands, and operates the walking assist device 3. Therefore, the grip handle 20L and the grip handle 20R are provided as a pair on left and right sides. Details of the grip handles 20L and 20R will be described below (see FIG. 2).

The grip handle 20L is held on an upper end of the handle frame 51L so as to extend rearward along the front-rear direction, and the wheel frame 52L is fixed to a lower side of the handle frame 51L. The handle frame 51L can extend and contract in an up-down direction, and a height of the grip handle 20L can be adjusted in accordance with a height of the hand of the user. The front wheel 60FL, which is a caster wheel capable of turning, is provided on a front side of the wheel frame 52L, and the rear wheel 60RL, which is driven by the travel drive device 64L, is provided on a rear side of the wheel frame 52L. The handle frame 51R, the grip handle 20R, the wheel frame 52R, the front wheel 60FR, the travel drive device 64R, and the rear wheel 60RR are also configured in the same manner.

The travel drive device 64L is, for example, an electric motor, and rotationally drives the rear wheel 60RL based on a control signal from the control device 40 based on electric power supplied from the battery B. Similarly, the travel drive device 64R is, for example, an electric motor, and rotationally drives the rear wheel 60RR based on the control signal from the control device 40 based on the electric power supplied from the battery B. The control device 40 is provided in the main frame 50.

The travel drive device 64L is provided with a travel speed detection device 64LE such as an encoder, and outputs a detection signal corresponding to rotation of the travel drive device 64L to the control device 40. The control device 40 can detect a travel speed of the walking assist device 3 relative to the ground (travel speed of the rear wheel 60RL) based on the detection signal from the travel speed detection device 64LE. Similarly, the travel drive device 64R is provided with a travel speed detection device 64RE such as an encoder, and outputs a detection signal corresponding to rotation of the travel drive device 64R to the control device 40. The control device 40 can detect the travel speed of the walking assist device 3 relative to the ground (travel speed of the rear wheel 60RR) based on the detection signal from the travel speed detection device 64RE.

The handle cover 30L is formed in a substantially box shape so as to cover an upper end portion of the handle frame 51L and an operation force direction detection device 71L which is attached to an upper end portion of a right side wall portion 51LR of the handle frame 51L on a side facing the handle frame 51R. The handle cover 30R is formed in a substantially box shape so as to cover an upper end portion of the handle frame 51R and an operation force direction detection device 71R which is attached to an upper end portion of a left side wall portion 51RL of the handle frame 51R on a side facing the handle frame 51L. Details of the operation force direction detection devices 71L and 71R will be described later below (see FIG. 2).

A main switch 12 is provided on a rear end portion of an upper end surface of the handle cover 30R. The main switch 12 is a switch that instructs activation of the walking assist device 3. When the user turns on the main switch 12, electric power is supplied from the battery B to the control device 40 and the travel drive devices 64R and 64L so as to enable operations, driving and traveling of the walking assist device 3.

An attachment member 15 to which the smartphone 5 is detachably attached is provided on a front side end edge portion of the upper end surface of the handle cover 30R. The attachment member 15 extends forward and obliquely upward from the front side end edge portion of the upper end surface of the handle cover 30R, and is formed in a substantially shallow groove shape whose left-right direction cross section has a width that is substantially the same as a left-right direction width of the smartphone 5. The smartphone 5 is fitted to the attachment member 15 from above and is detachably fixed by screwing or the like.

The grip handle 20L is a portion to be gripped by the left hand of the user, and protrudes rearward from the upper end portion of the handle frame 51L. As will be described later below, the grip handle 20L is movable relative to the handle frame 51L (that is, relative to the main frame 50) in a direction of an operation force from a neutral position when the user does not operate to a position near the neutral position (for example, about 1 mm), that is, in the main frame front-rear direction (see FIG. 2).

Similarly, the grip handle 20R is a portion to be gripped by the right hand of the user, and protrudes rearward from the upper end portion of the handle frame 51R. The grip handle 20R is movable relative to the handle frame 51R (that is, relative to the main frame 50) in a direction of an operation force from a neutral position when the user does not operate to a position near the neutral position (for example, about 1 mm), that is, in the main frame front-rear direction.

The operation force direction detection device 71L outputs, to the control device 40, a detection signal corresponding to movement of the grip handle 20L in the front-rear direction caused by the operation force of the user from the neutral position of the grip handle 20L when the user does not operate the grip handle 20L. The control device 40 can detect a direction of the operation force acting on the grip handle 20L relative to the handle frame 51L (that is, relative to the main frame 50) based on the detection signal from the operation force direction detection device 71L. That is, the control device 40 can detect whether the user pushes the grip handle 20L forward, releases the grip handle 20L to the neutral position, or pulls the grip handle 20L rearward based on the detection signal from the operation force direction detection device 71L.

Similarly, the operation force direction detection device 71R outputs, to the control device 40, a detection signal corresponding to movement of the grip handle 20R in the front-rear direction caused by the operation force of the user from the neutral position of the grip handle 20R when the user does not operate the grip handle 20R. The control device 40 can detect a direction of the operation force acting on the grip handle 20R relative to the handle frame 51R (that is, relative to the main frame 50) based on the detection signal from the operation force direction detection device 71R. That is, the control device 40 can detect whether the user pushes the grip handle 20R forward, releases the grip handle 20R to the neutral position, or pulls the grip handle 20R rearward based on the detection signal from the operation force direction detection device 71R.

A 3-axis acceleration and angular velocity sensor 50S is provided on the main frame 50 to measure acceleration relative to each axis in three directions of the X-axis, the Y-axis, and the Z axis, measure angular velocity of rotation about each axis in the three directions, and output a detection signal based on a result of the measurement to the control device 40. For example, when the walking assist device 3 travels on an inclined surface, the 3-axis acceleration and angular velocity sensor 50S outputs, to the control device 40, a detection signal corresponding to an inclination angle of the walking assist device 3 relative to each of the X-axis, the Y-axis, and the Z-axis.

[Detailed Structures of Grip Handle 20L and Operation Force Direction Detection Device 71L]

Next, detailed structures of the grip handles 20L and 20R and the operation force direction detection devices 71L and 71R will be described with reference to FIG. 2. As shown in FIG. 1, the grip handles 20L and 20R and the operation force direction detection devices 71L and 71R are arranged in pairs on left and right sides and symmetrically in the left-right direction. Therefore, the grip handle 20L and the operation force direction detection device 71L on the left side will be described as an example, and descriptions of the grip handle 20R and the operation force direction detection device 71R on the right side will be omitted.

As shown in FIG. 2, the grip handle 20L includes a shaft member 22L, a slide member 23L, a grip member 25L, and the like. A cylindrical attachment cylindrical portion BKL1 of the brake lever BKL is attached to a base end portion side of the shaft member 22L in a state of being abutted against the handle frame 51L. A pair of left and right frame side guide grooves 28 which are recessed in a rectangular shape in a front view are provided on two side wall portions in the left-right direction of the upper end portion of the handle frame 51L.

The pair of left and right frame side guide grooves 28 are formed with a through hole (insertion hole) 32 which penetrates the handle frame 51L in the left-right direction at a front-rear direction center position thereof and has a rectangular cross section. An up-down direction height of the cross section of the through hole 32 is substantially equal to a diameter of a support shaft (shaft member) 33 which has a circular cross section. A front-rear direction width of the through hole 32 is formed to be larger than the diameter of the support shaft 33, and the support shaft 33 is fitted and inserted thereto so as to be movable by a predetermined dimension (for example, about 2 mm to 3 mm) in the front-rear direction relative to a central axis 32B of the through hole 32.

When the slide member 23L which is formed in a substantially cylindrical shape is inserted and arranged, the slide member 23L is slidably supported on an outer side of the shaft member 22L. The slide member 23L includes a cylindrical portion 231 which is formed in a cylindrical shape, and a pair of left and right holding portions 232 formed in elongated plate shapes that extend from a front side tip end portion of the cylindrical portion 231 so as to face each other in a radial direction and extend toward radial direction outer sides.

The rubber grip member 25L, which is formed in a bottomed cylindrical shape whose rear side is closed, is fitted to and mounted on an outer side of the cylindrical portion 231 of the slide member 23L from a rear end side.

A pair of left and right through holes 232A, which are formed to have an inner diameter that is substantially equal to an outer diameter of the support shaft 33, are coaxially provided in tip end portions of the pair of left and right holding portions 232 so as to face each other. An up-down direction width of the pair of left and right holding portions 232 is substantially the same as an up-down direction width of the pair of left and right frame side guide grooves 28 formed in the upper end portion of the handle frame 51L.

When the cylindrical portion 231 is arranged on the outer side of the shaft member 22L, each of the pair of left and right holding portions 232 is inserted into a gap formed between the shaft member 22L and an inner peripheral surface of the attachment cylindrical portion BKL1 of the brake lever BKL in a manner that allows sliding in the front-rear direction, and is further inserted into the pair of left and right frame side guide grooves 28 formed in the upper end portion of the handle frame 51L in a manner that allows sliding in the front-rear direction.

The pair of left and right through holes 232A formed in the tip end portions of the pair of left and right holding portions 232 face each other with the through hole 32, which penetrates the pair of left and right frame side guide grooves 28 in the left-right direction and has the rectangular cross section, interposed therebetween. Then the support shaft (shaft member) 33, which has the circular cross section, is fitted in the left-right direction from one through hole 232A to the other through hole 232A via the through hole 32.

As shown in FIG. 2, a pair of front and rear spring recessed portions 38, which are formed with substantially the same rectangular cross section and open upward, are recessed along the up-down direction in a left-right direction center of an upper end surface of the handle frame 51L. The pair of spring recessed portions 38 are arranged along the front-rear direction at an interval that is substantially equal to the diameter of the support shaft 33 or an interval that is slightly smaller than the diameter of the support shaft 33 (for example, 0.3 mm to 1.0 mm smaller) with the central axis 32B of the through hole 32 interposed therebetween at a front-rear direction center. A bottom surface portion of each of the pair of spring recessed portions 38 is located below a predetermined height (for example, a height substantially equal to a radius of the support shaft 33) below a bottom surface of the through hole 32 which has the rectangular cross section.

A left-right direction width of a rectangular cross section of each spring recessed portion 38 is formed to be slightly larger (for example, about 0.3 mm to 0.6 mm larger) than an outer diameter of a pair of compression coil springs 39 which are pushed into each spring recessed portion 38 from above. The outer diameter of the pair of compression coil springs 39 is larger than the diameter of the support shaft 33, for example, the outer diameter is formed to be about 2 to 3 times the diameter of the support shaft 33. A front-rear direction width of the rectangular cross section of each spring recessed portion 38 is slightly shorter than an entire length of the compression coil spring 39 (for example, about 0.5 mm to 2 mm shorter). The pair of compression coil springs 39 have the same shape and are set to the same spring constant.

Accordingly, each of the pair of compression coil springs 39 is pushed into each spring recessed portion 38 from above in a compressed state, and is abutted against the bottom surface portion of each spring recessed portion 38. As a result, each compression coil spring 39 which is pushed to a back side of each of the pair of spring recessed portions 38 is abutted against an outer peripheral surface of the support shaft (shaft member) 33 which is fitted into the through hole 32, and biases the support shaft 33 from two sides in the front-rear direction such that the support shaft 33 is located at a center position between the pair of spring recessed portions 38, that is, on the central axis 32B of the through hole 32.

A gap 43 is formed between the tip end portion of each holding portion 232 of the slide member 23L and a front side inner wall surface of each frame side guide groove 28. A front-rear direction distance of the gap 43 is set to be larger (for example, about 1 mm to 2 mm larger) than a distance by which the support shaft 33 is movable in the front-rear direction from the state where the support shaft 33 is located on the central axis 32B of the through hole 32.

As a result, when the user grips the grip member 25L and pushes the grip member 25L forward, the support shaft 33 can be moved to a distance, by which the support shaft 33 is allowed to move forward, from the state of being located on the central axis 32B of the through hole 32 against a biasing force of the compression coil spring 39. When the user grips the grip member 25L and pulls the grip member 25L rearward, the support shaft 33 can be moved to a distance, by which the support shaft 33 is allowed to move rearward, from the state of being located on the central axis 32B of the through hole 32 against the biasing force of the compression coil spring 39.

Next, a schematic configuration of the operation force direction detection device 71L will be described with reference to FIG. 2. As shown in FIG. 2, the operation force direction detection device 71L includes a base member 72, a moving member 73, a pair of pressure sensitive sensors (moving direction detection members) 75, and the like. The base member 72 includes a flat plate portion 72A which has a rectangular shape in a front view and is attached to cover the frame side guide groove 28 formed in the upper end portion of the right side wall portion 51LR (see FIG. 1) on a left-right direction inner side of the handle frame 51L, and a pair of flange portions 72B which have a substantially rectangular shape and extend from two front-rear direction side edge portions of the flat plate portion 72A in a substantially right-angled rightward direction, that is, in a substantially right-angled inward direction over an entire length of the flat plate portion 72A.

In the flat plate portion 72A, a stopper through hole 72C, which has a rectangular cross section and penetrates coaxially with the central axis 32B of the through hole 32, is formed at a position facing the through hole 32 formed in the frame side guide groove 28. Accordingly, the support shaft 33, which is fitted into the stopper through hole 72C, is configured to be movable in the front-rear direction from a neutral position when the support shaft 33 is located on the central axis 32B of the through hole 32.

The moving member 73 is formed in a substantially rectangular parallelepiped shape that is slidably abutted against the flat plate portion 72A of the base member 72. A pair of stepped portions 73A that are recessed leftward, that is, toward the flat plate portion 72A, are formed on two front-rear direction end portions of the moving portion 73. A pair of sensor abutment portions 73B, which are formed in a substantially cylindrical shape by an elastic body such as silicon rubber, for example, are coaxially attached to the two front-rear direction end portions of the moving member 73 so as to protrude outward in the front-rear direction from the moving member 73. The moving member 73 is fixed, by an E-ring 78, to a right side end portion of the support shaft 33 which protrudes from the stopper through hole 72C. Meanwhile, the E-ring 78 is fixed to a left side end portion of the support shaft 33. The support shaft 33 is made of metal such as stainless steel, and the E-ring 78 is a plate-shaped member which is made of metal.

The pair of pressure sensitive sensors 75 are attached to front-rear direction inner side surfaces of the flange portions 72B of the base member 72 by adhesion or the like so as to face the pair of sensor abutment portions 73B of the moving member 73 in the front-rear direction. Each pressure sensitive sensor 75 is an element that electrically detects pressure. For example, the pressure sensitive sensor 75 is formed of a pressure sensitive resistance film which includes an electrode layer and a pressure sensitive resistance layer.

When no operation force is detected, that is, when the support shaft 33 and the moving member 73 are located at neutral positions while the pressure sensitive sensor 75 is not pressed by the sensor abutment portion 73B, the electrode layer and the pressure sensitive resistance layer are not in contact with each other and are thus in an insulated state, and a resistance value of the pressure sensitive sensor 75 increases (for example, several MS2 or more). On the other hand, when the operation force is detected, that is, when the support shaft 33 and the moving member 73 move in a moving direction while the pressure sensitive sensor 75 is pressed by the sensor abutment portion 73B, the electrode layer and the pressure sensitive resistance layer are brought into contact with each other, and electric resistance of the pressure sensitive sensor 75 is detected corresponding to pressure. As the pressure increases, the resistance value of the pressure sensitive sensor 75 decreases (to about several kΩ).

As a result, the operation force direction detection device 71L outputs, to the control device 40, a detection signal of each pressure sensitive sensor 75 corresponding to front-rear direction movement of the support shaft 33 and the moving member 73 caused by the operation force of the user from the neutral positions of the support shaft 33 and the moving member 73 when the user does not operate the grip handle 20L.

That is, the control device 40 can detect whether the user pushes the grip handle 20L forward, releases the grip handle 20L (neutral), or pulls the grip handle 20L rearward based on the detection signal of each pressure sensitive sensor 75 from the operation force direction detection device 71L. Similarly, the control device 40 can detect whether the user pushes the grip handle 20R forward, releases the grip handle 20R (neutral), or pulls the grip handle 20R rearward based on the detection signal of each pressure sensitive sensor 75 from the operation force direction detection device 71R.

[Control Configuration of Walking Assist Device 3]

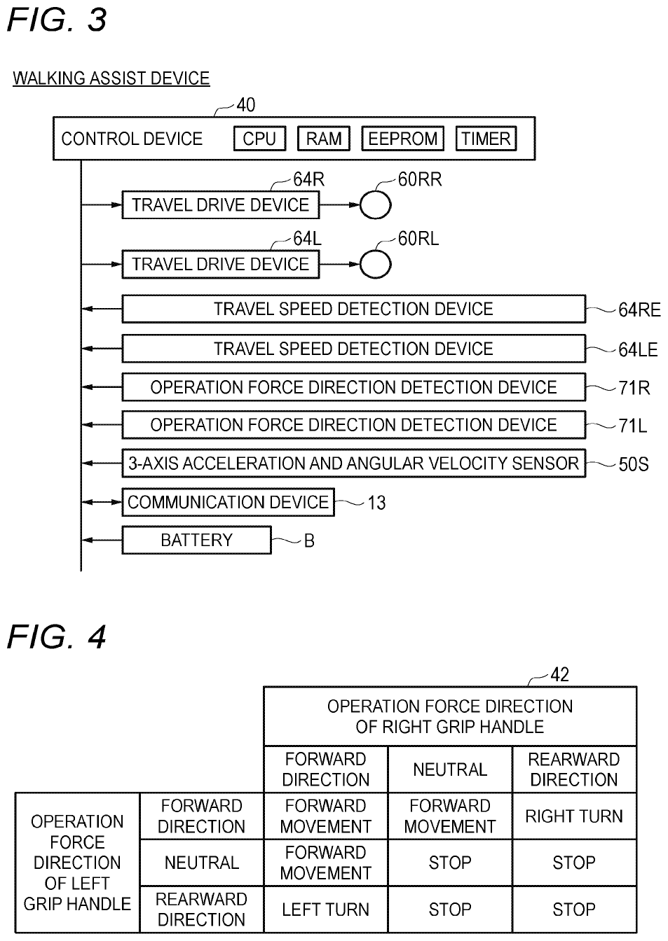

Next, a control configuration of the walking assist device 3 will be described with reference to FIG. 3. As shown in FIG. 3, the control device 40 is a known device including a CPU, an EEPROM, a RAM, a timer, and the like. The CPU executes various calculation processes based on various programs and various parameters stored in the EEPROM. The RANI temporarily stores calculation results of the CPU, data input from each detection device, and the like.

Detection signals from the travel speed detection devices 64LE and 64RE, detection signals from the operation force direction detection devices 71L and 71R, and detection signals from the 3-axis acceleration and angular velocity sensor 50S are input to the control device 40. A battery remaining amount (SOC) detection signal from a battery remaining amount detection device (not shown) provided in the battery B is also input to the control device 40. The control device 40 also outputs a control signal to the travel drive devices 64L and 64R.

A communication device 13 (see FIG. 1) is electrically connected to the control device 40. The control device 40 can transmit and receive information data to and from the smartphone 5 by wireless communication via the communication device 13 through using Bluetooth (registered trademark), Wi-Fi (registered trademark), or the like.

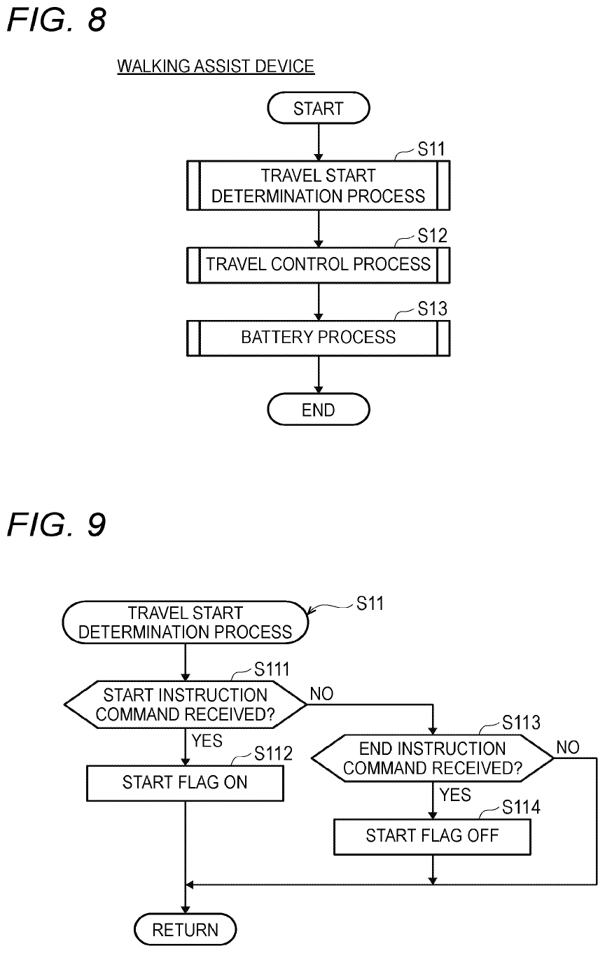

Here, for example, a drive table 42 shown in FIG. 4 is stored in advance in the EEPROM of the control device 40. The drive table 42 is used when the control device 40 determines a traveling-or-turning mode in a “travel control process” (see FIG. 8) to be described later below. As shown in FIG. 4, the drive table 42 stores one of “forward movement”, “stop”, “right turn”, and “left turn” in accordance with a combination of “forward direction”, “neutral”, and “rearward direction” of an “operation force direction of the left grip handle” and an “operation force direction of the right grip handle” corresponding to the detection signals input from the operation force direction detection devices 71L and 71R.

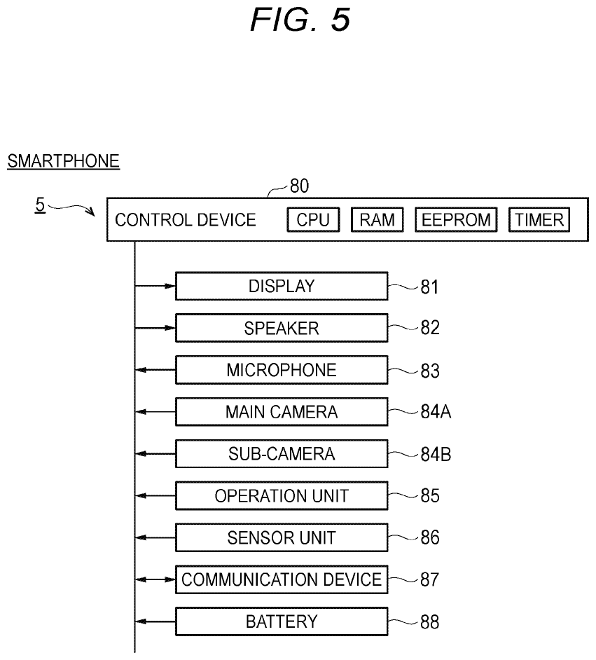

For example, in a case where the “operation force direction of the left grip handle” and the “operation force direction of the right grip handle” are both the “forward direction”, the traveling-or-turning mode of “forward movement” is stored in the drive table 42. In a case where the “operation force direction of the left grip handle” is the “forward direction” while the “operation force direction of the right grip handle” is the “rearward direction”, the traveling-or-turning mode of “right turning” is stored in the drive table 42.

[Schematic Configuration of Smartphone 5] Next, a schematic configuration of the smartphone 5 will be described with reference to FIGS. 5 to 7. As shown in FIG. 5, the smartphone 5 is a known device, and includes a control device 80, a display 81 including a liquid crystal display or the like, a speaker 82, a microphone 83, a main camera 84A arranged on a back face side of the display 81, a sub-camera 84B arranged on a screen side of the display 81, an operation unit 85 including an operation button or a touch panel that covers a screen of the display 81, a sensor unit 86 including a GPS sensor or the like, a communication device 87 capable of wireless communication, a battery 88, and the like.

The control device 80 is a known device including a CPU, an EEPROM, a RAM, a timer, and the like. The CPU executes various calculation processes based on various programs and various parameters stored in the EEPROM. The RANI temporarily stores calculation results of the CPU, data received via the communication device 87, and the like.

A voice detection signal from the microphone 83, a captured image signal from each of the cameras 84A and 84B, an operation signal corresponding to an operation of the user on the operation button or the touch panel from the operation unit 85, a current position detection signal of the GPS sensor from the sensor unit 86, and the like are input to the control device 80. A battery remaining amount (SOC) detection signal from a battery remaining amount detection device (not shown) provided in the battery 88 is also input to the control device 80.

The communication device 87 is electrically connected to the control device 80. The control device 80 can transmit and receive information data to and from the control device 40 of the walking assist device 3 or a server connected to the Internet by wireless communication via the communication device 87 through using Bluetooth (registered trademark), Wi-Fi (registered trademark), or the like. The control device 80 also outputs an image display signal for displaying images such as various icons and various input buttons on the display 81, and outputs a drive signal for outputting a voice or a sound to the speaker 82.

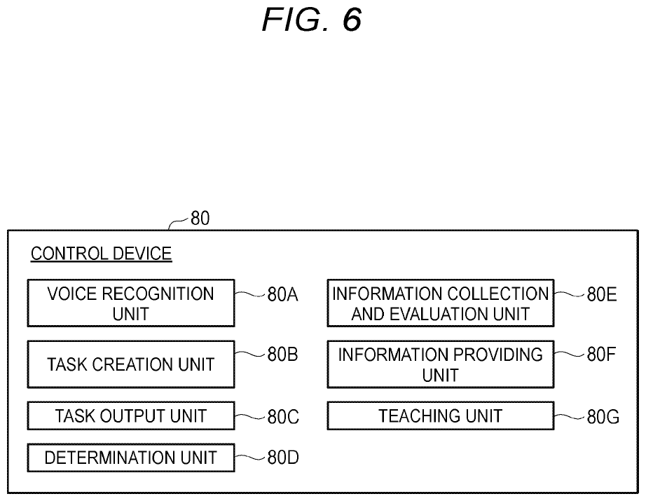

As shown in FIG. 6, the control device 80 includes, which will be described later below, a voice recognition unit 80A, a task creation unit 80B, a task output unit 80C (task load unit), a determination unit 80D (answer analysis unit), an information collection and evaluation unit 80E, an information providing unit 80F, a teaching unit 80G, and the like.

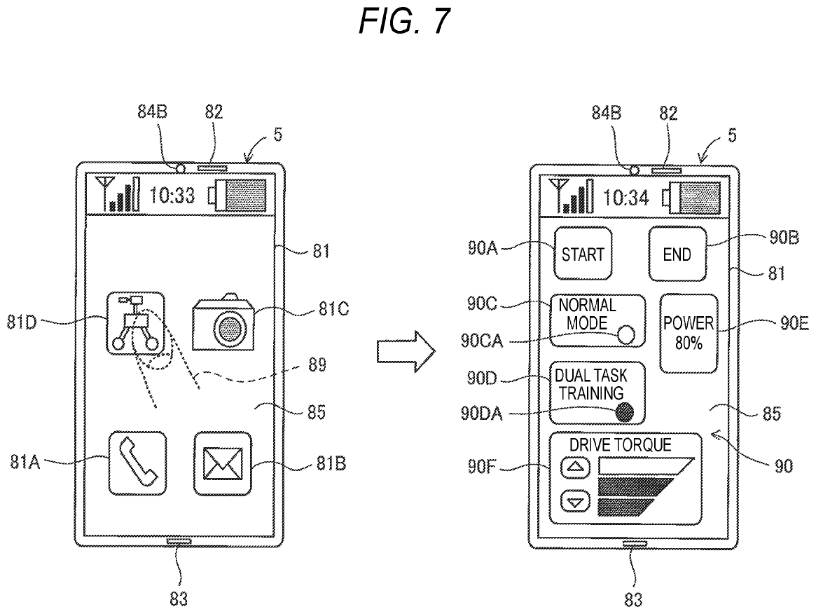

Here, an example of a display screen displayed on the display 81 by the control device 80 will be described with reference to FIG. 7. As shown in FIG. 7, in the smartphone 5, the speaker 82 and the sub-camera 84B are arranged on an upper side of an upper end edge portion of the display 81, while the microphone 83 is arranged on a lower side of a lower end edge portion of the display 81. The operation unit 85 configured by the touch panel covers the entire display screen of the display 81.

The control device 80 displays an antenna display portion indicating a reception state of the communication device 87, a display portion of time and the battery remaining amount (SOC) of the battery 88, and the like on the upper end edge portion of the display 81. As shown on a left side of FIG. 7, the control device 80 displays a telephone icon 81A, a mail icon 81B, a camera icon 81C, a walking training icon 81D, and the like on an initial screen of the display 81.

When the user presses the telephone icon 81A with a finger 89, the control device 80 starts and executes a telephone application program that causes the smartphone 5 to function as a telephone. When the user presses the mail icon 81B with the finger 89, the control device 80 starts and executes a mail application program that functions to enable transmission and reception of e-mail. When the user presses the camera icon 81C with the finger 89, the control device 80 starts and executes a camera application program that causes the smartphone 5 to function as a camera.

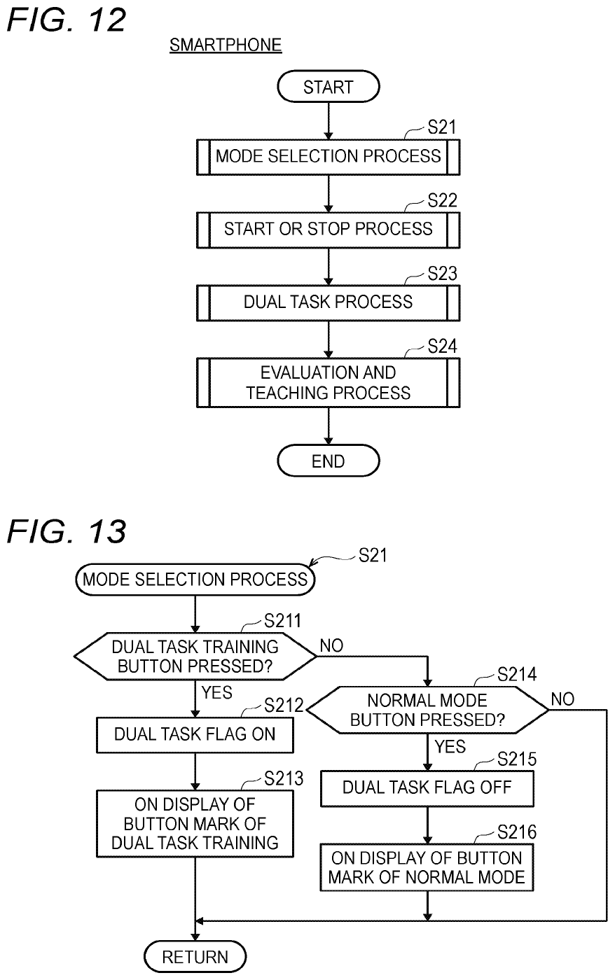

As shown on the left side of FIG. 7, when the user presses the walking training icon 81D with the finger 89, for example, as shown on a right side of FIG. 7, the control device 80 causes the smartphone 5 to function as an operation panel of the walking assist device 3, and starts and executes a walking support application program which includes a program of a first walking training process (see FIG. 12) to be described later below. Specifically, as shown on the right side of FIG. 7, the control device 80 displays, on the display 81, an operation panel screen 90 where a start button 90A, an end button 90B, a normal mode button 90C, a dual task training button 90D, a battery remaining amount display portion 90E, a drive torque adjustment portion 90F, and the like are displayed. The control device 80 also detects whether each of the buttons 90A to 90D is pressed by the finger 89 via the operation unit 85 configured by the touch panel or the like.

For example, when the start button 90A displayed on the display 81 is pressed, travel control of the walking assist device 3 is started. When the end button 90B is pressed, the travel control of the walking assist device 3 is stopped. When the normal mode button 90C is pressed, walking support is set in a normal mode, that is, the walking support during which the walking assist device 3 travels in accordance with the operation force directions of the left and right grip handles 20L and 20R is set. When the dual task training button 90D is pressed, the walking support is set in a dual task training mode, that is, the walking support during which the walking assist device 3 travels in accordance with the operation force directions of the left and right grip handles 20L and 20R while a dual task (double task) such as calculation and speaking words that need to be recalled is simultaneously performed is set.

A battery remaining amount of the battery B of the walking assist device 3 is displayed on the battery remaining amount display portion 90E. The drive torque adjustment unit 90F is an input unit configured for the user to adjust strength of drive torque of the travel drive devices 64L and 64R when the walking assist device 3 travels. For example, when the walking assist device 3 is used on an ascending slope, the user inputs an instruction to increase the drive torque from the drive torque adjustment unit 90F.

[Details of Walking Training Control]

Next, a processing procedure performed by each of the control devices 40 and 80 when the user selects and performs one of the normal walking training and the dual task (double task) training through using the walking support system for combined training 1 configured as described above will be described with reference to FIGS. 8 to 17.

Specifically, first, the user turns on the main switch 12 (see FIG. 1) of the walking assist device 3 configured as described above. Moreover, the user activates the smartphone 5 detachably attached to the attachment member 15 (see FIG. 1) of the walking assist device 3, and presses the walking training icon 81D displayed on the initial screen of the display 81 with the finger 89 (see FIG. 7).

When the user turns on the main switch 12, the control device 40 of the walking assist device 3 is activated to execute a “walking support process” shown in FIG. 8 at predetermined time intervals (for example, intervals of several [ms]), and the process proceeds to step S11. When the user presses the walking training icon 81D with the finger 89, the control device 80 of the smartphone 5 displays the operation panel screen 90 (see FIG. 7) on the display 81. Thereafter, the control device 80 executes the “first walking training process” shown in FIG. 12 at predetermined time intervals (for example, intervals of several [ms]), and the process proceeds to step S21. As a result, the user can start one of the normal walking training or the dual task (double task) training through using the walking support system for combined training 1.

[Walking Support Process]

The “walking support process” executed by the control device 40 of the walking assist device 3 will be described with reference to FIGS. 8 to 11. Programs shown in flowcharts of FIGS. 8 to 11 are stored in advance in the EEPROM of the control device 40.

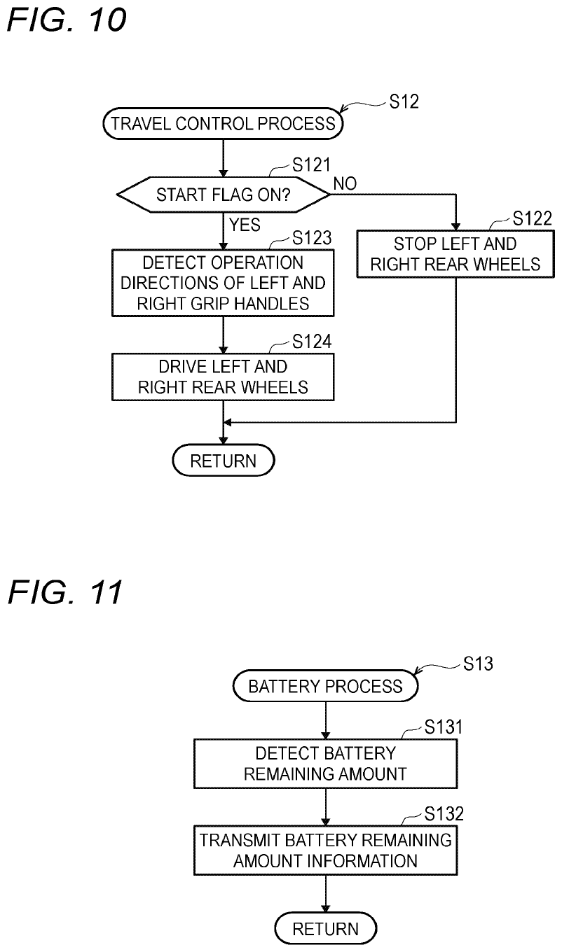

As shown in FIG. 8, first, in step S11, the control device 40 executes a sub-process of a “travel start determination process” (see FIG. 9) to be described later below, and then proceeds to step S12. In step S12, the control device 40 executes a sub-process of the “travel control process” (see FIG. 10), and then proceeds to step S13. In step S13, the control device 40 executes a sub-process of a “battery process” (see FIG. 11), and then ends the walking support process.

[Travel Start Determination Process]

Next, the sub-process of the “travel start determination process” will be described with reference to FIG. 9. As shown in FIG. 9, first, in step S111, the control device 40 determines whether a “start instruction command” is received from the smartphone 5 via the communication device 13. The “start instruction command” is a command that instructs the travel drive devices 64L and 64R to be driven in accordance with the combination of the operation force directions of the left and right grip handles 20L and 20R operated by the user and to start traveling in accordance with walking of the user.

When it is determined that the “start instruction command” is received from the smartphone 5 via the communication device 13 (S111: YES), the control device 40 proceeds to step S112. In step S112, the control device 40 reads a start flag from the RAM, sets the start flag to ON, stores the start flag in the RAM again, and then ends the sub-process and proceeds to the sub-process of the “travel control process” of step S12. The start flag is set to OFF and stored in the RAM when the control device 40 is activated.

On the other hand, when it is determined that the “start instruction command” is not received from the smartphone 5 via the communication device 13 (S111: NO), the control device 40 proceeds to step S113. In step S113, the control device 40 determines whether an “end instruction command” is received from the smartphone 5 via the communication device 13. The “end instruction command” is a command that instructs to stop driving of the travel drive devices 64L and 64R and to end (stop) the traveling in accordance with the walking of the user.

When it is determined that the “end instruction command” is received from the smartphone 5 via the communication device 13 (S113: YES), the control device 40 proceeds to step S114. In step S114, the control device 40 reads the start flag from the RAM, sets the start flag to OFF, stores the start flag in the RANI again, and then ends the sub-process and proceeds to the sub-process of the “travel control process” of step S12.

On the other hand, when it is determined that the “end instruction command” is not received from the smartphone 5 via the communication device 13 (S113: NO), the control device 40 ends the sub-process and proceeds to the sub-process of the “travel control process” of step S12. That is, the control device 40 proceeds to the sub-process of the “travel control process” of step S12 without changing the start flag.

[Travel Control Process]

Next, the sub-process of the “travel control process” will be described with reference to FIG. 10. As shown in FIG. 10, first, in step S121, the control device 40 reads the start flag from the RAM, and determines whether the start flag is set to ON, that is, whether the start instruction command is received from the smartphone 5.

When it is determined that the start flag read from the RAM is set to OFF (S121: NO), the control device 40 determines that the start instruction command is not received from the smartphone 5 or the end instruction command is received from the smartphone 5, and proceeds to step S122. In step S122, the control device 40 stops the driving of the travel drive devices 64L and 64R to stop the left and right rear wheels 60RL and 60RR, and then ends the sub-process and proceeds to the sub-process of the “battery process” of step S13.

On the other hand, when it is determined that the start flag read from the RAM is set to ON (S121: YES), the control device 40 determines that the start instruction command is received from the smartphone 5, and proceeds to step S123. In step S123, the control device 40 detects, based on the detection signals of the pressure sensitive sensors 75 (see FIG. 2) from the operation force direction detection devices 71L and 71R, whether the user pushes the left and right grip handles 20L and 20R forward, releases the left and right grip handles 20L and 20R to the neutral position, or pulls the left and right grip handles 20L and 20R rearward, stores a result of the detection in the RAM, and then proceeds to step S124. That is, the control device 40 detects the operation force directions of the left and right grip handles 20L and 20R, stores the detected operation force directions in the RAM, and then proceeds to step S124.

In step S124, the control device 40 reads the operation force directions of the left and right grip handles 20L and 20R from the RAM, and sets the operation force directions as the “operation force direction of the left grip handle” and the “operation force direction of the right grip handle” of the drive table 42 (see FIG. 4). Then the control device 40 reads one traveling-or-turning mode among the “forward movement”, “stop”, “right turn”, and “left turn” from the drive table 42 according to the combination of the operation force directions of the “forward direction”, “neutral”, and “rearward direction”, and stores the traveling-or-turning mode in the RAM.

Then the control device 40 outputs the control signal to each of the travel drive devices 64L and 64R in accordance with the traveling-or-turning mode read from the drive table 42, drives the left and right rear wheels 60RL and 60RR for a predetermined time (for example, several milliseconds), and then ends the sub-process and proceeds to the sub-process of the “battery process” of step S13. For example, when the traveling-or-turning mode is “forward movement”, the control device 40 outputs the control signal of “forward movement” to each of the travel drive devices 64L and 64R, drives the left and right rear wheels 60RL and 60RR to rotate for a predetermined time (for example, several milliseconds) in a forward direction, and then ends the sub-process and proceeds to the sub-process of the “battery process” of step S13.

For example, when the traveling-or-turning mode is “right turn”, the control signal of “forward movement” is output to the left travel drive device 64L, the control signal of “stop” is output to the right travel drive device 64R, the right rear wheel 60RR is stopped while only the left rear wheel 60RL is driven for a predetermined time (for example, several milliseconds), and then the sub-process is ended and the process proceeds to the sub-process of the “battery process” of step S13. For example, when the traveling-or-turning mode is “stop”, the control device 40 outputs the control signal of “stop” to each of the travel drive devices 64L and 64R, stops the left and right rear wheels 60RL and 60RR for a predetermined time (for example, several milliseconds), and then ends the sub-process and proceeds to the sub-process of the “battery process” of step S13.

[Battery Process]

Next, the sub-process of the “battery process” will be described with reference to FIG. 11. As shown in FIG. 11, first, in step S131, the control device 40 detects the battery remaining amount of the battery B based on the battery remaining amount (SOC) detection signal input from the battery remaining amount detection device (not shown) provided in the battery B, stores the detected battery remaining amount in the RAM, and then proceeds to step S132. In step S132, the control device 40 reads the battery remaining amount of the battery B from the RAM, transmits battery remaining amount information including data of the battery remaining amount to the smartphone 5 via the communication device 13, and then ends the sub-process and ends the walking support process.

[First Walking Training Process]

Next, the “first walking training process” executed by the control device 80 of the smartphone 5 will be described with reference to FIGS. 12 to 17. Programs shown in flowcharts of FIGS. 12 to 16 are stored in advance in the EEPROM of the control device 80.

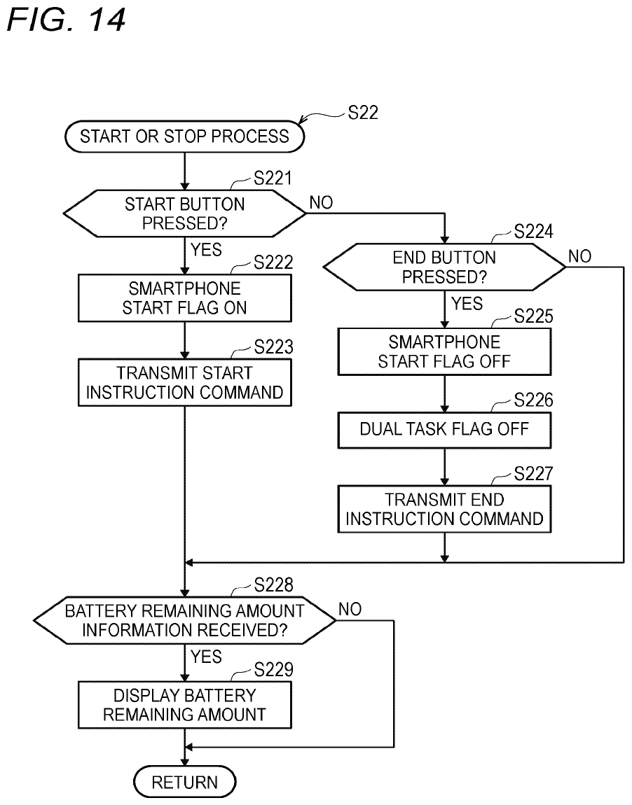

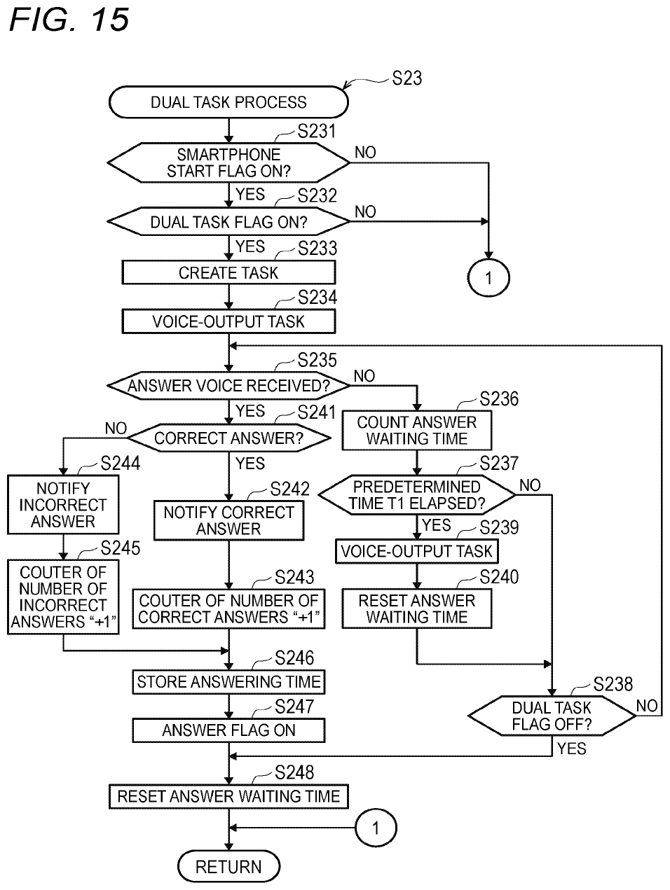

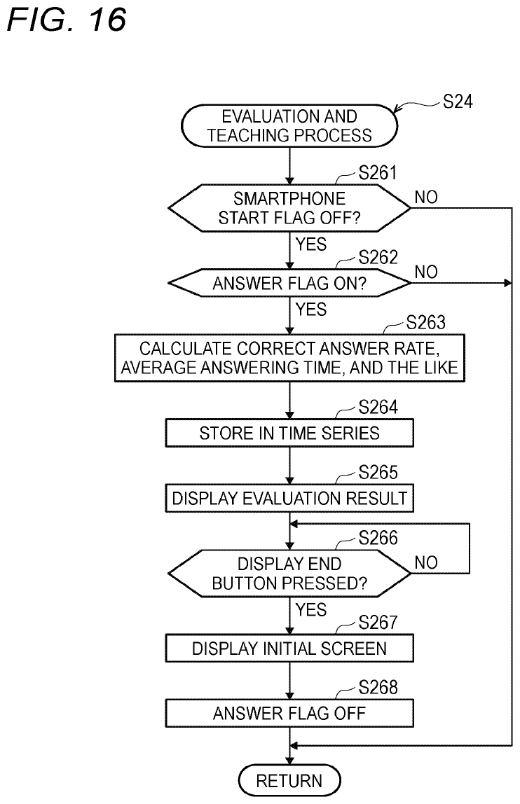

As shown in FIG. 12, first, in step S21, the control device 80 executes a sub-process of a “mode selection process” (see FIG. 13) to be described later below, and then proceeds to step S22. In step S22, the control device 80 executes a sub-process of a “start or stop process” (see FIG. 14), and then proceeds to step S23. In step S23, the control device 80 executes a sub-process of a “dual task process” (see FIG. 15), and then proceeds to step S24. In step S24, the control device 80 executes a sub-process of an “evaluation and teaching process” (see FIG. 16), and then ends the first walking training process.

[Mode Selection Process]

Next, the sub-process of the “mode selection process” will be described with reference to FIG. 13. As shown in FIG. 13, first, in step S211, the control device 80 determines whether the dual task training button 90D (see FIG. 7) of the operation panel screen 90 (see FIG. 7) of the operation panel screen 90 (see FIG. 7) displayed on the display 81 is pressed by the finger 89 (see FIG. 7). When the user selects the walking support of the dual task training mode, the user presses the dual task training button 90D (see FIG. 7). On the other hand, when the user selects the walking support of the normal mode, the user presses the normal mode button 90C (see FIG. 7).

When it is determined that the dual task training button 90D is pressed by the finger 89 (S211: YES), the control device 80 proceeds to step S212. In step S212, the control device 80 reads a dual task flag from the RAM, sets the dual task flag to ON, stores the dual task flag in the RAM again, and then proceeds to step S213. The dual task flag is set to OFF and stored in the RAM when the control device 80 is activated.

In step S213, the control device 80 sets a button mark 90DA (see FIG. 7) marked with a white circle on the dual task training button 90D to ON display (for example, display of blinking red), and then ends the sub-process and proceeds to the sub-process of the “start or stop process” of step S22. As a result, the fact that the walking support of the dual training mode is selected is displayed. When the button mark 90DA marked with the white circle on the dual task training button 90D is set to the ON display, the control device 80 sets a button mark 90CA (see FIG. 7) of the normal mode button 90C to OFF display (for example, display of the white circle).

On the other hand, when it is determined in S211 that the dual task training button 90D is not pressed by the finger 89 (S211: NO), the control device 80 proceeds to step S214. In step S214, the control device 80 determines whether the normal mode button 90C of the operation panel screen 90 displayed on the display 81 is pressed by the finger 89. When it is determined that the normal mode button 90C is not pressed by the finger 89 (S214: NO), the control device 80 ends the sub-process and proceeds to the sub-process of the “start or stop process” of step S22.

On the other hand, when it is determined that the normal mode button 90C of the operation panel screen 90 displayed on the display 81 is pressed by the finger 89 (S214: YES), the control device 80 proceeds to step S215. In step S215, the control device 80 reads the dual task flag from the RAM, sets the dual task flag to OFF, stores the dual task flag in the RAM again, and then proceeds to step S216.

In step S216, the control device 80 sets the button mark 90CA marked with the white circle on the normal mode button 90C to the ON display (for example, the display of the blinking red), and then ends the sub-process and proceeds to the sub-process of the “start or stop process” of step S22. As a result, the fact that the walking support of the normal mode is selected is displayed. When the button mark 90CA marked with the white circle on the normal mode button 90C is set to the ON display, the control device 80 sets the button mark 90DA of the dual task training button 90D to the OFF display (for example, the display of the white circle).

[Start or Stop Process]

Next, the sub-process of the “start or stop process” will be described with reference to FIG. 14. As shown in FIG. 14, first, in step S221, the control device 80 determines whether the start button 90A (see FIG. 7) of the operation panel screen 90 (see FIG. 7) displayed on the display 81 is pressed by the finger 89 (see FIG. 7). When walking is started during the walking support of the normal mode or the walking support of the dual task training mode, the user presses the start button 90A with the finger 89.

When it is determined that the start button 90A is pressed by the finger 89 (S221: YES), the control device 80 proceeds to step S222. In step S222, the control device 80 reads a smartphone start flag from the RAM, sets the smartphone start flag to ON, stores the smartphone start flag in the RAM again, and then proceeds to step S223. The smartphone start flag is set to OFF and stored in the RAM when the control device 80 is activated.

In step S223, the control device 80 transmits the “start instruction command”, which instructs the walking assist device 3 to start traveling in accordance with the walking of the user, to the walking assist device 3 via the communication device 87, and then proceeds to step S228 to be described later below.

On the other hand, when it is determined in step S221 that the start button 90A is not pressed by the finger 89 (S221: NO), the control device 80 proceeds to step S224. In step S224, the control device 80 determines whether the end button 90B (see FIG. 7) of the operation panel screen 90 (see FIG. 7) displayed on the display 81 is pressed by the finger 89 (see FIG. 7). When the walking is ended during the walking support of the normal mode or the walking support of the dual task training mode, the user presses the end button 90B with the finger 89.

When it is determined that the end button 90B is not pressed by the finger 89 (S224: NO), the control device 80 proceeds to step S228 to be described later below. As a result, when the user is walking during the walking support of the normal mode or the walking support of the dual task training mode, the walking is continued in such a state.

On the other hand, when it is determined that the end button 90B is pressed by the finger 89 (S224: YES), the control device 80 proceeds to step S225. In step S225, the control device 80 reads the smartphone start flag from the RAM, sets the smartphone start flag to OFF, stores the smartphone start flag in the RAM again, and then proceeds to step S226. In step S226, the control device 80 reads the dual task flag from the RAM, sets the dual task flag to OFF, stores the dual task flag in the RAM again, and then proceeds to step S227.

In step S227, the control device 80 transmits the “end instruction command”, which instructs the walking assist device 3 to end (stop) the traveling in accordance with the walking of the user, to the walking assist device 3 via the communication device 87, and then proceeds to step S228. As a result, the walking assist device 3 is in a stopped state (see FIG. 10).

In step S228, the control device 80 determines whether the battery remaining amount information including the data of the battery remaining amount of the battery B is received from the walking assist device 3 via the communication device 87. When it is determined that the battery remaining amount information is not received from the walking assist device 3 (S228: NO), the control device 80 ends the sub-process and proceeds to the sub-process of the “dual task process” of step S23.

On the other hand, when it is determined that the battery remaining amount information is received from the walking assist device 3 (S228: YES), the control device 80 proceeds to step S229. In step S229, the control device 80 displays the battery remaining amount of the battery B included in the battery remaining amount information on the battery remaining amount display portion 90E of the operation panel screen 90 (see FIG. 7) displayed on the display 81, and then ends the sub-process and proceeds to the sub-process of the “dual task process” of step S23. For example, as shown in FIG. 7, when the battery remaining amount of the battery B included in the battery remaining amount information is “80%”, the control device 80 displays “power 80%” on the battery remaining amount display portion 90E of the operation panel screen 90.

[Dual Task Process]

Next, the sub-process of the “dual task process” will be described with reference to FIG. 15. As shown in FIG. 15, first, in step S231, the control device 80 reads the smartphone start flag from the RAM, and determines whether the smartphone start flag is set to ON. When it is determined that the smartphone start flag is set to OFF (S231: NO), the control device 80 ends the sub-process and proceeds to the sub-process of the “evaluation and teaching process” of step S24.

On the other hand, when it is determined that the smartphone start flag is set to ON (S231: YES), the control device 80 determines that the start button 90A (see FIG. 7) of the operation panel screen 90 is pressed, and proceeds to step S232. In step S232, the control device 80 reads the dual task flag from the RAM, and determines whether the dual task flag is set to ON. When it is determined that the dual task flag is set to OFF (S232: NO), the control device 80 ends the sub-process and proceeds to the sub-process of the “evaluation and teaching process” of step S24.

On the other hand, when it is determined that the dual task flag is set to ON (S231: YES), the control device 80 determines that the dual task training button 90D of the operation panel screen 90 is pressed, and proceeds to step S233. In step S233, the control device 80 creates or selects a task to be spoken via the speaker 82 to the user who uses the walking assist device 3 to walk, stores the task in the RAM, and then proceeds to step S234. Accordingly, processing of step S233 executed by the control device 80 corresponds to the task creation unit 80B.

The task to be spoken to the user includes at least one of a calculation question to be answered by the user through performing mental arithmetic of calculation (for example, what is the total number of legs of three birds and one cat?), a four-basic-math-operation question to be answered through performing mental arithmetic of four basic math operations (for example, how many will there be left if “7” is subtracted from “100”, and how many will there be left if another “7” is subtracted therefrom?), a quiz question (for example, what month and day is it today?), a question that need to be recalled by the user such as a question of a menu of a meal eaten by the user (for example, what did you eat for dinner yesterday?), and the like.

For example, a plurality of the calculation questions, a plurality of the four-basic-math-operation questions, a plurality of the quiz questions, and a plurality of the questions of the meal menu may be stored in the EEPROM in advance, and the control device 80 may randomly select one question from the plurality of questions. Answers to the calculation questions, the four-basic-math-operation questions, and the quiz questions are also stored in the EEPROM in advance.

The user may capture a photograph of a dish by the main camera 84A each time the user eats a meal, and the control device 80 may store image data of the dish photograph captured by the main camera 84A in the EEPROM together with date and time. Then the control device 80 may create or select the question of the meal menu relative to a recent dish photograph based on the stored image data of a plurality of the dish photographs. The control device 80 may create an answer to the question of the meal menu by analyzing the image data of the recent dish photograph, and store the answer in the RAM.

Next, in step S234, the control device 80 reads the task stored in the RAM in step S233, voice-outputs the task via the speaker 82, asks the walking user about the task, and then proceeds to step S235. In step S235, the control device 80 determines whether an answer voice of the user to the task voice-output in step S234 is received via the microphone 83. Accordingly, processing of step S234 executed by the control device 80 corresponds to the task output unit 80C.

When it is determined that the answer voice to the task is not received via the microphone 83 (S235: NO), the control device 80 proceeds to step S236. In step S236, the control device 80 counts a waiting time for the answer from the user, stores the waiting time in the RAM, and then proceeds to step S237. In step S237, the control device 80 reads the answer waiting time from the RAM, and determines whether the answer waiting time reaches a predetermined time T1 (for example, T1=20 seconds) (first elapsed time), that is, whether the predetermined time T1 has elapsed.

When it is determined that the answer waiting time does not reach the predetermined time T1 (for example, T1=20 seconds), that is, the predetermined time T1 has not elapsed (S237: NO), the control device 80 proceeds to step S238. In step S238, the control device 80 reads the dual task flag from the RAM, and determines whether the dual task flag is set to OFF, that is, whether the end button 90B (see FIG. 7) of the operation panel screen 90 is pressed.

When it is determined that the dual task flag is set to ON, that is, the end button 90B of the operation panel screen 90 is not pressed (S238: NO), the control device 80 executes processing of step S235 and subsequent steps again. On the other hand, when it is determined that the dual task flag is set to OFF, that is, the end button 90B of the operation panel screen 90 is pressed (S238: YES), the control device 80 proceeds to step S248 to be described later below.

On the other hand, when it is determined in step S237 that the answer waiting time reaches the predetermined time T1 (for example, T1=20 seconds), that is, the predetermined time T1 has elapsed (S237: YES), the control device 80 proceeds to step S239. In step S239, the control device 80 reads the task stored in the RAM in step S233, voice-outputs the task via the speaker 82, asks the walking user about the task again, and then proceeds to step S240. In step S240, the control device 80 reads the answer waiting time from the RAM, resets the answer waiting time, stores the answer waiting time in the RAM again, and then executes processing of step S238 and subsequent steps again.

As a result, even if the user fails to hear the task voice-output via the speaker 82 in step S234, the user can hear the task again. In step S239, the control device 80 may also execute processing of step S233 and step S234, change the task spoken via the speaker 82, voice-output the changed task to the user who uses the walking assist device 3 to walk and question the user about the task. As a result, even if the user does not know the answer to the task and thus cannot answer, the user can answer the task questioned again.

On the other hand, when it is determined that the answer voice of the user to the task is received via the microphone 83 in step S235 (S235: YES), the control device 80 performs a voice recognition process on the answer voice, analyzes answered contents thereof, stores the answered contents in the RAM, and then proceeds to step S241. Accordingly, processing of step S235 executed by the control device 80 corresponds to the voice recognition unit 80A.

In step S241, the control device 80 reads the answer to the task created in step S233 from the EEPROM or the RAM, and determines whether the answered contents of the user received via the microphone 83 in step S235 is correct. When it is determined that the answered contents of the user received via the microphone 83 is correct (S241: YES), the control device 80 proceeds to step S242. Accordingly, processing of step S241 executed by the control device 80 corresponds to the determination unit 80D.

In step S242, the control device 80 notifies the user that the answer is correct (analysis information), and then proceeds to step S243. For example, the control device 80 outputs an answer-correct sound such as “Pinpoon” via the speaker 82 to notify that the answer is correct. The control device 80 may also display a large white circle on the display 81 to notify that the answer is correct.

In step S243, the control device 80 reads a count value of a counter of the number of correct answers obtained by counting the number of correct answers from the RAM, adds “1” to the count value, stores the count value in the RAM again, and then proceeds to step S246 to be described later below. The count value of the counter of the number of correct answers is reset and stored in the RAM when the control device 80 is activated.

On the other hand, when it is determined that the answered contents of the user received via the microphone 83 in step S241 is incorrect (S241: NO), the control device 80 proceeds to step S244. In step S244, the control device 80 notifies the user that the answer is incorrect (analysis information), and then proceeds to step S245. For example, the control device 80 outputs an answer-incorrect sound such as “Boo-boo” via the speaker 82 to notify that the answer is incorrect. The control device 80 may also display a large X mark on the display 81 to notify that the answer is incorrect.

In step S245, the control device 80 reads a count value of a counter of the number of incorrect answers obtained by counting the number of incorrect answers from the RAM, adds “1” to the count value, stores the count value in the RAM again, and then proceeds to step S246. The count value of the counter of the number of incorrect answers is reset and stored in the RAM when the control device 80 is activated.

In step S246, the control device 80 reads the answer waiting time from the RAM, stores the answer waiting time in the RANI in time series as an “answering time” relative to the current task, and then proceeds to step S247. In step S247, the control device 80 reads an answer flag from the RAM, sets the answer flag to ON, stores the answer flag in the RAM again, and then proceeds to step S248. The answer flag is set to OFF and stored in the RAM when the control device 80 is activated.

In step S248, the control device 80 reads the answer waiting time from the RAM, resets the answer waiting time, stores the answer waiting time in the RAM again, and then ends the sub-process and proceeds to the sub-process of the “evaluation and teaching process” of step S24.

[Evaluation and Teaching Process]

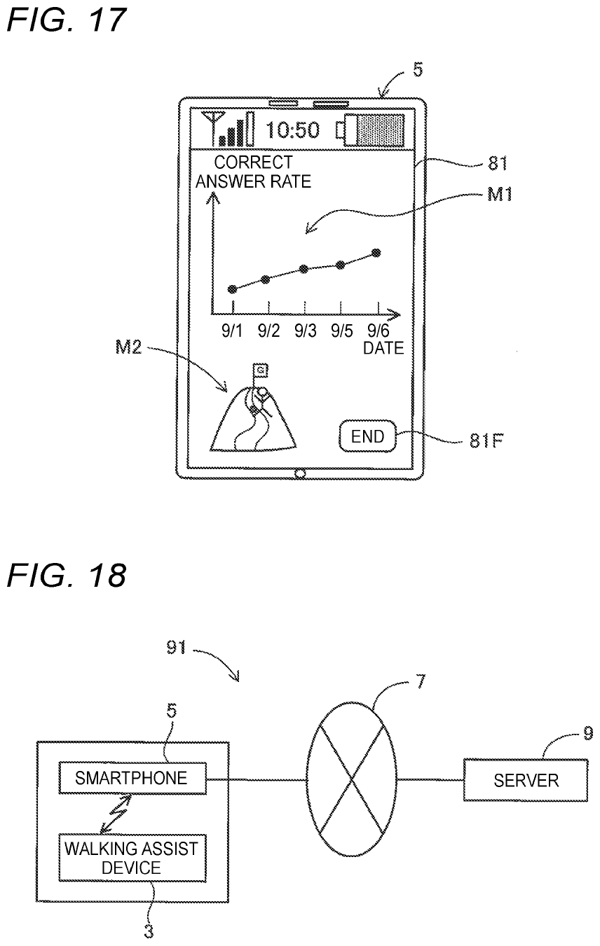

Next, the sub-process of the “evaluation and teaching process” will be described with reference to FIGS. 16 and 17. As shown in FIG. 16, first, in step S261, the control device 80 reads the smartphone start flag from the RAM, and determines whether the smartphone start flag is set to OFF. When it is determined that the smartphone start flag is set to ON (S261: NO), the control device 80 determines that the end button 90B (see FIG. 7) of the operation panel screen 90 is not pressed, ends the sub-process, and ends the first walking training process.

On the other hand, when it is determined that the smartphone start flag is set to OFF (S261: YES), the control device 80 determines that the end button 90B of the operation panel screen 90 is pressed and the walking assist device 3 is stopped, and proceeds to step S262.

In step S262, the control device 80 reads the answer flag from the RAM, and determines whether the answer flag is set to ON. When it is determined that the answer flag is set to OFF (S262: NO), the control device 80 determines that the answer voice is not received from the user via the microphone 83, ends the sub-process, and ends the first walking training process.

On the other hand, when it is determined that the answer flag is set to ON (S262: YES), the control device 80 determines that the answer voice is received from the user via the microphone 83, and proceeds to step S263. In step S263, the control device 80 calculates the number of correct answers among the answers answered by the user to the task voice-output via the speaker 82, a correct answer rate, a level of achievement relative to a target correct answer rate, an average answering time, and the like, and then proceeds to step S264.

Specifically, the control device 80 reads the count value of the counter of the number of correct answers from the RANI, and calculates the count value as a current number of correct answers. The control device 80 also reads the count values of the counter of the number of incorrect answers and the counter of the number of correct answers from the RAM, divides the count value of the counter of the number of correct answers by a total count value of the counter of the number of incorrect answers and the counter of the number of correct answers so as to calculate the correct answer rate. The control device 80 also reads the target correct answer rate stored in advance from the EEPROM, divides the calculated correct answer rate by the target correct answer rate so as to calculate the level of achievement relative to the target correct answer rate.

In step S264, the control device 80 stores the calculated number of correct answers among the answers answered by the user, the correct answer rate, the level of achievement relative to the target correct answer rate, the average answering time, and the like in the EEPROM in time series together with information of date and time, and then proceeds to step S265. In step S265, the control device 80 reads, from the EEPROM, the number of correct answers among the answers answered by the user to the tasks of several days in the past, the correct answer rate, the level of achievement relative to the target correct answer rate, and the like, creates a graph or design for such parameters, displays the graphed or designed parameters on the display 81 as an evaluation result or a reward, and then proceeds to step S266. Accordingly, processing of steps S263 to S265 executed by the control device 80 corresponds to the information collection and evaluation unit 80E, the information providing unit 80F, and the teaching unit 80G.

For example, as shown in FIG. 17, the control device 80 reads the correct answer rate of the past five days from the EEPROM, creates a correct answer rate transition map M1, and displays the correct answer rate transition map M1 on the display 81 as the evaluation result. The control device 80 also reads the level of achievement relative to the current target correct answer rate from the EEPROM, creates a level-of-achievement map M2 in which a position of the user who climbs toward a mountain top goal indicates the level of achievement relative to the target correct answer rate, and displays the level-of-achievement map M2 on the display 81 as the reward for the user. The control device 80 also displays a display end button 81F on the display 81. When the display end button 81F is pressed, the control device 80 ends the display of the correct answer rate transition map M1, the level-of-achievement map M2, and the like, and displays the initial screen (see the left side of FIG. 7) of the display 81.

In step S266, the control device 80 waits for the display end button 81F displayed on the display 81 to be pressed (S266: NO). When the display end button 81F displayed on the display 81 is pressed (S266: YES), the control device 80 proceeds to step S267. In step S267, the control device 80 ends the display of the graph or the design of the number of correct answers among the answers answered by the user, the correct answer rate, the level of achievement relative to the target correct answer rate, and the like, displays the initial screen (see the left side of FIG. 7) of the display 81, and then proceeds to step S268.

In step S268, the control device 80 reads the answer flag from the RAM, sets the answer flag to OFF, stores the answer flag in the RAM again, ends the sub-process, and ends the first walking training process. As a result, the smartphone 5 is in a state where the initial screen (see the left side of FIG. 7) is displayed on the display 81, and the walking assist device 3 is in the stopped state and returned to an initial state.

Here, the communication device 13 functions as an example of a device communication device. The communication device 87 functions as an example of a terminal communication device. The smartphone 5 functions as an example of a mobile terminal. The control device 80 of the smartphone 5 functions as an example of a task load unit, an answer analysis unit, a notification unit, an analysis information collection unit, an answer evaluation unit, a teaching unit, a reward granting unit, an elapsed time measurement unit, and a time determination unit. The speaker 82 functions as an example of a voice output unit. The microphone 83 functions as an example of a voice input unit.

As described above in detail, in the walking support system for combined training 1 according to the first embodiment, the user turns on the main switch 12 of the walking assist device 3. The user also activates the smartphone 5 detachably attached to the attachment member 15 of the walking assist device 3, and presses the walking training icon 81D displayed on the initial screen of the display 81 with the finger 89. Then the user presses the normal mode button 90C on the operation panel screen 90 displayed on the display 81 of the smartphone 5, and then presses the start button 90A. As a result, the user can walk while gripping the left and right grip handles 20L and 20R of the walking assist device 3 and perform walking training in the normal mode.

The user presses the dual task training button 90D on the operation panel screen 90 displayed on the display 81 of the smartphone 5, and then presses the start button 90A. As a result, the user can answer by voice in consideration of the answer to the task each time the task is voice-output from the speaker 82 of the smartphone 5 while performing the walking training during which the user walks while gripping the left and right grip handles 20L and 20R of the walking assist device 3.

Accordingly, the user can perform the dual task training in which the walking training is performed at the same time while answering by voice in consideration of the answer to the task, and thus the walking training can be performed more effectively. The smartphone 5 can also function as a so-called smart speaker which is capable of conversing with the user.

When the end button 90B is pressed, the smartphone 5 displays (teaches) evaluation information (for example, the correct answer rate transition map M1 indicating a change in the correct answer rate for each day or the level-of-achievement map M2) related to an execution degree of the task executed by the user on the display 81. As a result, interest of the user in providing the voice answer to the task during the walking training can be effectively attracted by the evaluation information displayed on the display 81, and motivation for the user to continuously perform the dual task training can be increased.

### Second Embodiment

Next, a walking support system for combined training 91 according to a second embodiment will be described with reference to FIGS. 18 to 22. The same reference numerals as those of the walking support system for combined training 1 according to the first embodiment denote the same or corresponding parts as those of the walking support system for combined training 1 according to the first embodiment.

The walking support system for combined training 91 according to the second embodiment has substantially the same configuration as the walking support system for combined training 1 according to the first embodiment. However, as shown in FIG. 18, the walking support system for combined training 91 includes the walking assist device 3, the smartphone 5 detachably attached to the attachment member 15 of the walking assist device 3, and a server 9.

Similarly to the walking support system for combined training 1 according to the first embodiment, the walking assist device 3 and the smartphone 5 are configured to be capable of transmitting and receiving information data to and from each other by wireless communication through using Bluetooth, Wi-Fi, or the like. The smartphone 5 and the server 9 are also configured to be capable of transmitting and receiving information data to and from each other by wireless communication through using Wi-Fi or the like via a network 7 such as the Internet.

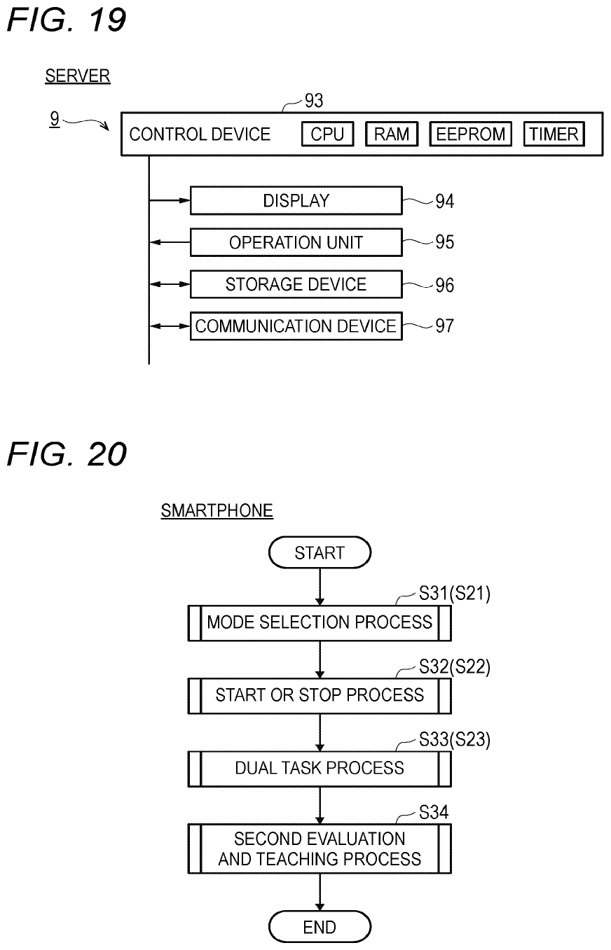

Next, a schematic configuration of the server 9 will be described with reference to FIG. 19. As shown in FIG. 19, the server 9 is a known device which includes a control device 93, a display 94 constituted by a liquid crystal display or the like, an operation unit 95 constituted by a keyboard, a mouse, and the like, a storage device 96 constituted by, for example, a hard disc drive (HDD) or a solid state drive (SSD), a communication device 97 capable of wirelessly communication through using Wi-Fi or the like, and the like.

The control device 93 is a known device including a CPU, an EEPROM, a RAM, a timer, and the like. The CPU executes various calculation processes based on various programs and various parameters stored in the EEPROM. The RAM temporarily stores calculation results of the CPU, data received via the communication device 97, and the like.

An operation signal corresponding to an operation of the user on the keyboard, the mouse, or the like from the operation unit 95 is input to the control device 93. The communication device 97 is electrically connected to the control device 93. The control device 93 can transmit and receive information data to and from the smartphone 5 or the like, which is connected to the network 7, via the communication device 97 by wireless communication through using Wi-Fi or the like. The control device 93 also outputs an image display signal for displaying, on the display 94, a message input from the keyboard or various types of image data received via the communication device 97. The control device 93 also stores various types of information data received via the communication device 97 and various calculated results in the storage device 96.

[Details of Walking Training Control]

Next, a processing procedure performed by each of the control devices 40, 80, and 93 when the user selects and performs one of the normal walking training and the dual task (double task) training through using the walking support system for combined training 91 configured as described above will be described with reference to FIGS. 20 to 22.

Specifically, first, the user turns on the main switch 12 (see FIG. 1) of the walking assist device 3 configured as described above. Moreover, the user activates the smartphone 5 detachably attached to the attachment member 15 (see FIG. 1) of the walking assist device 3, and presses the walking training icon 81D displayed on the initial screen of the display 81 with the finger 89 (see FIG. 7).

When the user turns on the main switch 12, the control device 40 of the walking assist device 3 is activated to execute the “walking support process” shown in FIG. 8 at the predetermined time intervals (for example, the intervals of several [ms]), and the process proceeds to step S11. When the user presses the walking training icon 81D with the finger 89, the control device 80 of the smartphone 5 displays the operation panel screen 90 (see FIG. 7) on the display 81. Thereafter, the control device 80 of the smartphone 5 executes a “second walking training process” (see FIG. 20) to be described later below instead of the “first walking training process” (see FIG. 12) at predetermined time intervals (for example, intervals of several [ms]).

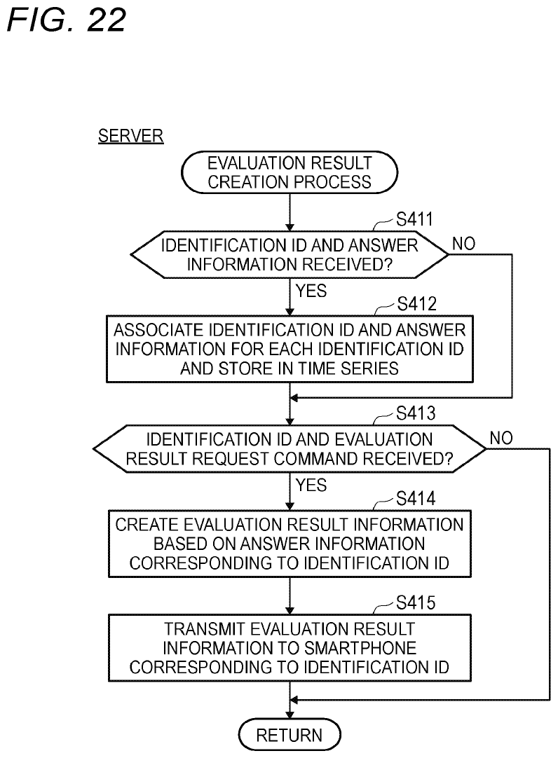

The control device 93 of the server 9 executes an “evaluation result creation process” (see FIG. 22) to be described later below at predetermined time intervals (for example, intervals of several [ms]). As will be described later below, when the control device 93 executes the “evaluation result creation process” (see FIG. 22) and receives an “evaluation result request command” from the smartphone 5, the control device 93 creates the evaluation information related to the execution degree of the task executed by the user, and transmits the evaluation information to the smartphone 5. The evaluation information includes, for example, the number of correct answers among the answers answered by the user to the task of the past several days as desired by the user or of preset past several days, the correct answer rate, the level of achievement relative to the target correct answer rate, and the average answering time.

[Second Walking Training Process]

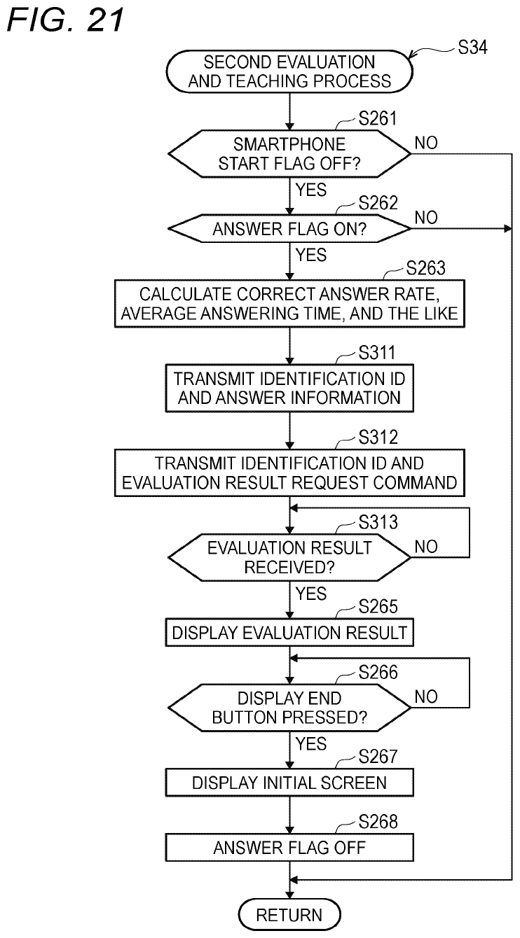

The “second walking training process” executed by the control device 80 of the smartphone 5 will be described with reference to FIGS. 20 and 21. Programs shown in flowcharts of FIGS. 20 and 21 are stored in advance in the EEPROM of the control device 80.

As shown in FIG. 20, in step S31, the control device 80 executes the sub-process of the “mode selection process” executed in step S21 of the “first walking training process” (see FIG. 12) described above, and then proceeds to step S32. In step S32, the control device 80 executes the sub-process of the “start or stop process” executed in step S22 described above, and then proceeds to step S33. In step S33, the control device 80 executes the sub-process of the “dual task process” executed in step S23 described above, and then proceeds to step S34. In step S34, the control device 80 executes the sub-process of a “second evaluation and teaching process” (see FIG. 21) instead of the “evaluation and teaching process”, and then ends the second walking training process.

[Second Evaluation and Teaching Process]

Next, the sub-process of the “second evaluation and teaching process” will be described with reference to FIG. 21. As shown in FIG. 21, the control device 80 first executes processing of steps S261 to S263 (see FIG. 16), and then proceeds to step S311.

In step S311, the control device 80 reads the count value of the counter of the number of correct answers, the count value of the counter of the number of incorrect answers, the average answering time, and the like from the RAM. Then the control device 80 creates answer information including the count value of the counter of the number of correct answers, the count value of the counter of the number of incorrect answers, the average answering time, and the like. Thereafter, the control device 80 adds an identification ID for identifying the smartphone 5 to the answer information, transmits the answer information to the server 9 via the communication device 87, and then proceeds to step S312. The identification ID for identifying the smartphone 5 is stored in the EEPROM in advance.

In step S312, the control device 80 adds the identification ID for identifying the smartphone 5 to the evaluation result request command that requests the number of correct answers among the answers answered by the user to the task of the past several days as desired or of the preset past several days, the correct answer rate, the level of achievement relative to the target correct answer rate, and the like, transmits the evaluation result request command to the server 9 via the communication device 87, and then proceeds to step S313.

In step S313, the control device 80 waits for evaluation result information (evaluation information) where the identification ID for identifying the smartphone 5 is added to be received from the server 9 via the communication device 87 in response to the evaluation result request command (S313: NO). The evaluation result information (evaluation information) includes, the number of correct answers among the answers answered by the user to the task of the past several days as desired or of the preset past several days, the correct answer rate, the level of achievement relative to the target correct answer rate, the average answering time, and the like. When the control device 80 receives the evaluation result information, where the identification ID for identifying the smartphone 5 is added, from the server 9 via the communication device 87 (S313: YES), the control device 80 stores the evaluation result information in the EEPROM, and then proceeds to step S265 (see FIG. 16).

Then the control device executes processing of steps S265 to S268 (see FIG. 16), and then ends the sub-process and ends the second walking training process. As a result, the smartphone 5 is in the state where the initial screen (see the left side of FIG. 7) is displayed on the display 81, and the walking assist device 3 is in the stopped state and returned to the initial state. Accordingly, processing of steps S263 to S265 executed by the control device 80 corresponds to the information providing unit 80F and the teaching unit 80G.

[Evaluation Result Creation Process]

Next, the “evaluation result creation process” executed by the control device 93 of the server 9 will be described with reference to FIG. 22. As shown in FIG. 22, first, in step S411, the control device 93 determines whether communication data, which is constituted by the “answer information” (including the count value of counter of the number of correct answers, the count value of the counter of the number of incorrect answers, the average answering time and the like) and the “identification ID” of the smartphone 5, is received via the communication device 97. When it is determined that the communication data constituted by the “answer information” and the “identification ID” of the smartphone 5 is not received via the communication device 97 (S411: NO), the control device 93 proceeds to step S413 to be described later below.

On the other hand, when it is determined that the communication data constituted by the “answer information” and the “identification ID” of the smartphone 5 is received via the communication device 97 (S411: YES), the control device 93 proceeds to step S412. In step S412, the control device 93 associates the “identification ID” and the “answer information” received via the communication device 97 for each identification ID for identifying the smartphone 5, stores the associated “identification ID” and “answer information” in the storage device 96 in time series together with information on date and time, and then proceeds to step S413. Accordingly, when the control device 93 receives the communication data constituted by the “answer information” and the “identification ID” from a plurality of the smartphones 5 via the communication device 97, the control device 93 stores each piece of “answer information” in the storage device 96 in correspondence with the “identification ID” of each smartphone 5.

In step S413, the control device 93 determines whether communication data constituted by the “evaluation result request command” that requests the number of correct answers among the answers answered by the user to the task of the past several days as desired or of the preset past several days, the correct answer rate, the level of achievement relative to the target correct answer rate, and the like, and the “identification ID” of the smartphone 5 is received via the communication device 97. When it is determined that the communication data constituted by the “evaluation result request command” and the “identification ID” of the smartphone 5 is not received via the communication device 97 (S413: NO), the control device 93 ends the process.

On the other hand, when it is determined that the communication data constituted by the “evaluation result request command” and the “identification ID” of the smartphone 5 is received via the communication device 97 (S413: YES), the control device 93 proceeds to step S414. In step S414, the control device 93 first reads, in accordance with the “evaluation result request command”, the “answer information” of the past several days as desired or of the preset past several days, which is stored in the storage device 96 in time series together with the information on date and time in association with the received identification ID of the smartphone 5, and stores the read “answer information” in the RAM. As described above, the “answer information” includes the count value of the counter of the number of correct answers, the count value of the counter of the number of incorrect answers, the average answering time, and the like.

Then the control device 93 calculates, in accordance with the “evaluation result request command”, the number of correct answers among the answers answered by the user to the task of the past several days as desired or of the preset past several days, the correct answer rate, the level of achievement relative to the target correct answer rate, the average answering time, and the like for each day. Then the control device 93 stores such calculation results in the RAM in time series together with respective date information as the “evaluation result information (evaluation information)”, and then proceeds to step S415.

Specifically, the control device 93 reads, from the RANI, the count value of the counter of the number of correct answers of the past several days as desired or of the preset past several days in accordance with the “evaluation result request command”, and calculates each count value as the number of correct answers for each date. The control device 93 also reads, from the RAM, a combination of the count values of the counter of the number of incorrect answers and the counter of the number of correct answers of the past several days as desired or of the preset past several days in accordance with the “evaluation result request command”.

Then the control device 93 divides the count value of the counter of the number of correct answers by the total count value of the counter of the number of incorrect answers and the counter of the number of correct answers for each date, so as to calculate the correct answer rate for each date. The control device 93 also reads the target correct answer rate stored in advance from the EEPROM or the storage device 96, divides the calculated correct answer rate for each day by the target correct answer rate so as to calculate the level of achievement relative to the target correct answer rate for each day.

In step S415, the control device 93 reads, from the RAM, “evaluation result information” of the past several days as desired or of the preset past several days in accordance with the “evaluation result request command”. Then the control device 93 adds the “identification ID” of the smartphone 5 received together with the “evaluation result request command” to the “evaluation result information (evaluation information)”, transmits the “evaluation result information (evaluation information)” to the smartphone 5 via the communication device 97, and then ends the process. Accordingly, processing of steps S411 to S415 executed by the control device 93 functions as the information collection and evaluation unit 80E of the control device 80 of the smartphone 5.

Here, the communication device 13 functions as an example of a device communication device. The communication device 87 functions as an example of a terminal communication device. The smartphone 5 functions as an example of a mobile terminal. The communication device 97 functions as an example of a server communication device. The control device 80 of the smartphone 5 functions as an example of a task load unit, an answer analysis unit, a notification unit, an analysis information collection unit, a teaching unit, a reward granting unit, an elapsed time measurement unit, a time determination unit, a terminal transmission unit, and a terminal reception unit. The speaker 82 functions as an example of a voice output unit. The microphone 83 functions as an example of a voice input unit. The control device 93 of the server 9 functions as an example of a server reception unit, an answer evaluation unit, and a server transmission unit.

As described above in detail, in the walking support system for combined training 91 according to the second embodiment, the user turns on the main switch 12 of the walking assist device 3. The user also activates the smartphone 5 detachably attached to the attachment member 15 of the walking assist device 3, and presses the walking training icon 81D displayed on the initial screen of the display 81 with the finger 89. Then the user presses the normal mode button 90C on the operation panel screen 90 displayed on the display 81 of the smartphone 5, and then presses the start button 90A. As a result, the user can walk while gripping the left and right grip handles 20L and 20R of the walking assist device 3 and perform walking training in the normal mode.

The user presses the dual task training button 90D on the operation panel screen 90 displayed on the display 81 of the smartphone 5, and then presses the start button 90A. As a result, the user can answer by voice in consideration of the answer to the task each time the task is voice-output from the speaker 82 of the smartphone 5 while performing the walking training during which the user walks while gripping the left and right grip handles 20L and 20R of the walking assist device 3. Accordingly, the user can perform the dual task training in which the walking training is performed at the same time while answering by voice in consideration of the answer to the task, and thus the walking training can be performed more effectively.

When the end button 90B is pressed, the smartphone 5 creates the answer information including the count value of the counter of the number of correct answers, the count value of the counter of the number of incorrect answers, the average answering time, and the like. Then the smartphone 5 adds the identification ID to the answer information and transmits the answer information to the server 9 via the communication device 87. The server 9 also associates the answer information received via the communication device 97 with the identification ID, and stores the answer information in the storage device 96 in time series together with the date information.

Thereafter, the smartphone 5 transmits the “evaluation result request command” that requests the number of correct answers among the answers answered by the user to the task of the past several days as desired or of the preset past several days, the correct answer rate, the level of achievement relative to the target correct answer rate, and the like, and the “identification ID” to the server 9 via the communication device 87. Then, when the server 9 receives the “evaluation result request command” and the “identification ID” via the communication device 97, the server 9 creates the “evaluation result information (evaluation information)” which includes the number of correct answers among the answers answered by the user to the task of the past several days as desired or of the preset past several days, the correct answer rate, the level of achievement relative to the target correct answer rate, and the like.

Then the server 9 adds the “identification ID” of the smartphone 5 to the “evaluation result information (evaluation information)” and transmits the “evaluation result information (evaluation information)” to the smartphone 5 via the communication device 97. Based on the “evaluation result information” received via the communication device 87, the smartphone 5 creates the evaluation information (for example, the correct answer rate transition map M1 indicating the change in the correct answer rate for each day or the level-of-achievement map M2) related to the execution degree of the task executed by the user, and displays (teaches) the evaluation information on the display 81.

As a result, the interest of the user in providing the voice answer to the task during the walking training can be effectively attracted by the evaluation information displayed on the display 81, and the motivation for the user to continuously perform the dual task training can be increased. Moreover, the server 9 creates the “evaluation result information” which includes the number of correct answers among the answers answered by the user to the task of the preset past several days, the correct answer rate, the level of achievement relative to the target correct answer rate, and the like, so that a processing load of the smartphone 5 can be reduced. Moreover, the server 9 can transmit the “evaluation result information” not only to the smartphone 5 but also to other processing devices such as personal computers.

### Third Embodiment

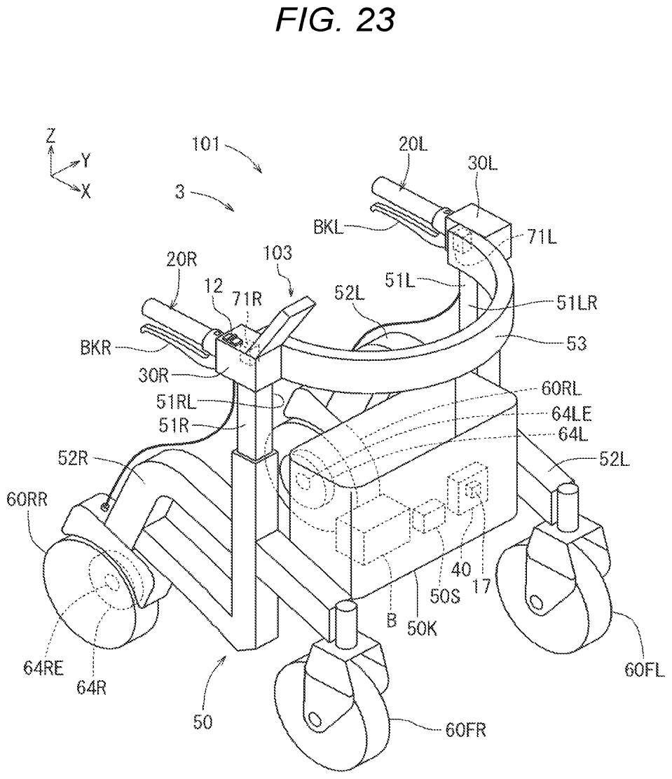

Next, a walking support system for combined training 101 according to a third embodiment will be described with reference to FIGS. 23 to 32. The same reference numerals as those of the walking support system for combined training 1 according to the first embodiment denote the same or corresponding parts as those of the walking support system for combined training 1 according to the first embodiment.

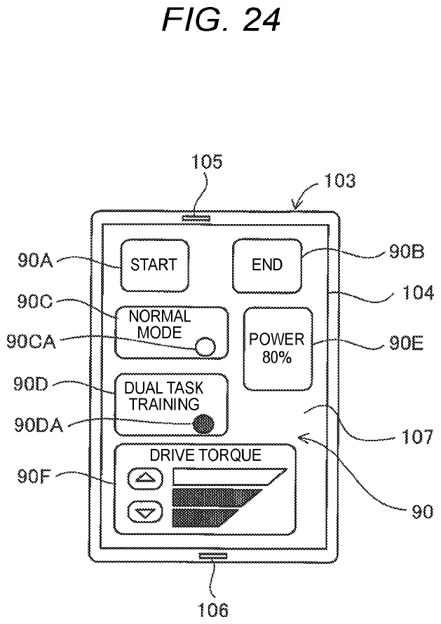

The walking support system for combined training 101 according to the third embodiment has substantially the same configuration as the walking support system for combined training 1 according to the first embodiment. However, as shown in FIG. 23, the walking support system for combined training 101 includes the walking assist device 3, and a display device 103 instead of the smartphone 5 and the attachment member 15. The display device 103 is attached so as to protrude forward and obliquely upward from the front side end edge portion of the upper end surface of the handle cover 30R, and is provided in such a manner that the user can easily view a display screen of a display 104 (see FIG. 24).

As shown in FIG. 24, the display device 103 includes the display 104 including a liquid crystal display or the like, a speaker 105 arranged on an upper side of an upper end edge portion of the display 104, a microphone 106 arranged on a lower side of a lower end edge portion of the display 104, an operation unit 107 including a touch panel or the like that covers the entire display screen of the display 104, and the like.

[Control Configuration of Walking Support System for Combined Training 101]

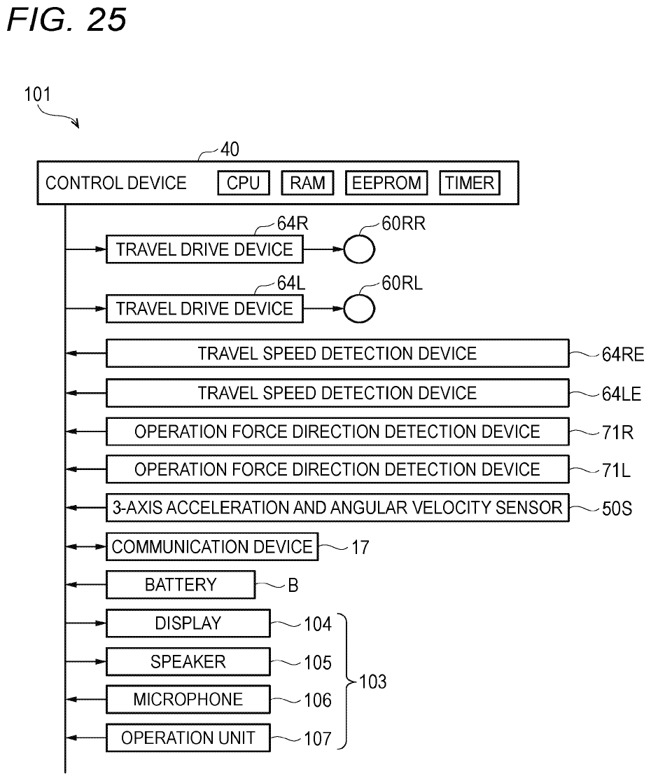

As shown in FIG. 25, a control configuration of the walking support system for combined training 101 is substantially the same as a control configuration of the walking support system for combined training 1 according to the first embodiment. Accordingly, the control device 40 provided on the main frame 50 of the walking assist device 3 drives and controls the entire walking support system for combined training 101.

However, as shown in FIG. 25, a communication device 17 is electrically connected to the control device 40 instead of the communication device 13 (see FIG. 1). The control device 40 can be connected to the network 7 (see FIG. 18), such as the Internet, via the communication device 17 through using Wi-Fi (registered trademark) or the like. In a case where the control device 40 does not communicate with any external device, the communication device 17 may not be provided in the walking assist device 3.

The display 104, the speaker 105, the microphone 106, the operation unit 107, and the like that constitute the display device 103 are electrically connected to the control device 40. A voice detection signal from the microphone 106, an operation signal corresponding to an operation of the user on the touch panel or the like from the operation unit 107, and the like are input to the control device 40. The control device 40 also outputs an image display signal for displaying images such as various icons and various input buttons on the display 104, and outputs a drive signal for outputting a voice or a sound to the speaker 105.

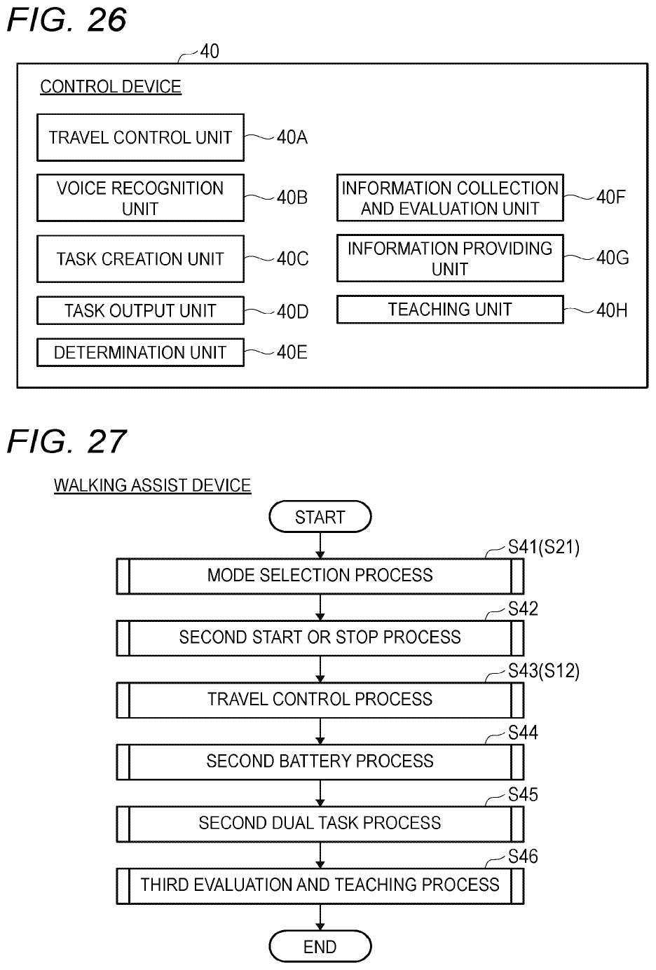

As shown in FIG. 26, the control device 40 includes, which will be described later below, a travel control unit 40A, a voice recognition unit 40B, a task creation unit 40C, a task output unit 40D (task load unit), a determination unit 40E (answer analysis unit), an information collection and evaluation unit 40F, an information providing unit 40G, a teaching unit 40H, and the like.

Here, when the user turns on the main switch 12 (see FIG. 23), the control device 40 is activated to execute a program of a third walking training process to be described later below (see FIG. 27) at predetermined time intervals (for example, intervals of several [ms]). The control device 40 also displays the operation panel screen 90 (see FIG. 24), which is displayed on the display 81 of the smartphone 5, on the display 104 of the display device 103 such that the display device 103 functions as an operation panel.

The control device 40 also detects whether each of the buttons 90A to 90D is pressed by the finger 89 (see FIG. 7) via the operation unit 107 configured by the touch panel or the like. Accordingly, when the start button 90A displayed on the display 104 is pressed, the travel control of the walking assist device 3 is started. When the end button 90B is pressed, the travel control of the walking assist device 3 is stopped. When the normal mode button 90C is pressed, the walking support is set in the normal mode. When the dual task training button 90D is pressed, the walking support is set in the dual task training mode.

[Third Walking Training Process]

Next, the “third walking training process” executed by the control device 40 of the walking assist device 3 will be described with reference to FIGS. 27 to 32. Programs shown in flowcharts of FIGS. 27 to 31 are stored in advance in the EEPROM of the control device 40.

As shown in FIG. 27, first, in step S41, the control device 40 executes the sub-process of the “mode selection process” (see FIGS. 12 and 13) executed by the control device 80 of the smartphone 5, and then proceeds to step S42. When the user selects the walking support of the dual task training mode, the user presses the dual task training button 90D (see FIG. 24) displayed on the display device 103. On the other hand, when the user selects the walking support of the normal mode, the user presses the normal mode button 90C (see FIG. 24) displayed on the display device 103.

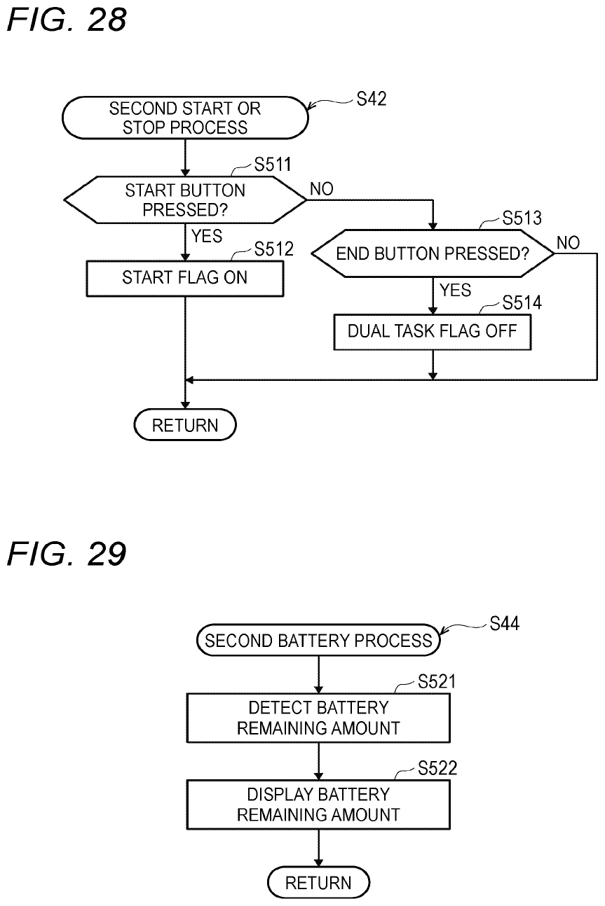

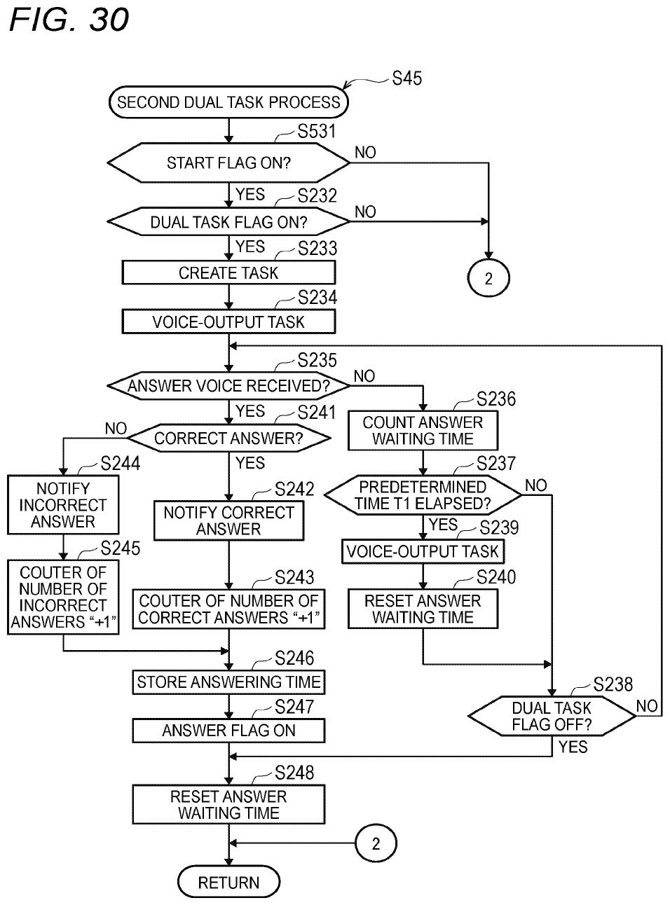

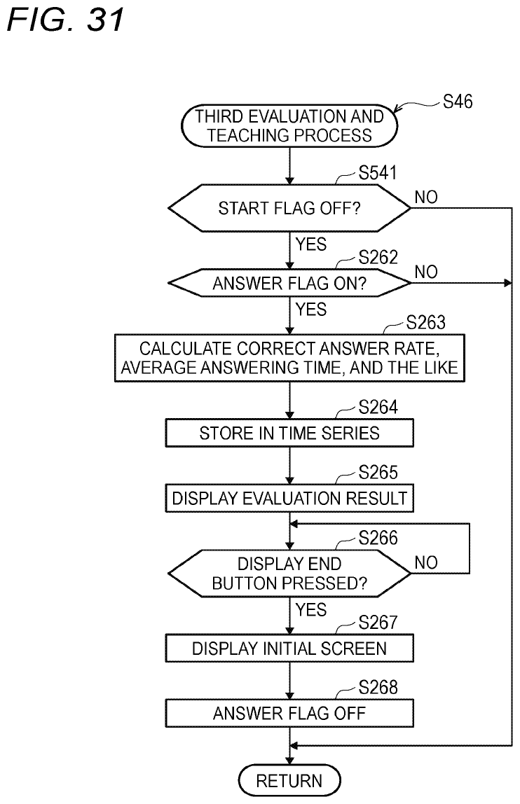

In step S42, the control device 40 executes a sub-process of a “second start or stop process” (see FIG. 28). Then, in step S43, the control device 40 executes the sub-process of the “travel control process” (see FIG. 8) executed in step S12 of the “walking support process” (see FIG. 8) described above. Accordingly, processing of step S43 corresponds to the travel control unit 40A. Subsequently, in step S44, the control device 40 executes a sub-process of a “second battery process” (see FIG. 29), and then executes a sub-process of a “second dual task process” (see FIG. 30) in step S45. Thereafter, in step S46, the control device 40 executes a sub-process of a “third evaluation and teaching process” (see FIG. 31), and then ends the third walking training process.

[Second Start or Stop Process]

Next, the sub-process of the “second start or stop process” will be described with reference to FIG. 28. As shown in FIG. 28, when the start button 90A (see FIG. 24) of the operation panel screen 90 (see FIG. 24) displayed on the display 104 is pressed by the finger 89 (see FIG. 7) (S511: YES), the control device 40 sets the start flag to ON (S512), ends the sub-process, and proceeds to the sub-process of the “travel control process” of step S43. The start flag is set to OFF and stored in the RAM when the control device 40 is activated.

On the other hand, when the end button 90B (see FIG. 24) is pressed by the finger 89 (see FIG. 7) (S511: NO to S513: YES), the control device 40 sets the start flag to OFF (S513), ends the sub-process, and proceeds to the sub-process of the “travel control process” of step S43. On the other hand, when the start button 90A and the end button 90B (see FIG. 24) are not pressed by the finger 89 (see FIG. 7) (S511: NO to S513: NO), the control device 40 ends the sub-process and proceeds to the sub-process of the “travel control process” of step S43.

[Second Battery Process]

Next, the sub-process of the “second battery process” will be described with reference to FIG. 29. As shown in FIG. 29, in step S521, the control device 40 detects the battery remaining amount of the battery B based on the battery remaining amount (SOC) detection signal input from the battery remaining amount detection device (not shown) provided in the battery B. Then, in step S522, the control device 40 displays the battery remaining amount on the battery remaining amount display portion 90E (see FIG. 24), and then ends the sub-process and proceeds to the sub-process of the “second dual task process” of step S45.

[Second Dual Task Process]

Next, the sub-process of the “second dual task process” will be described with reference to FIG. 30. As shown in FIG. 30, first, in step S531, the control device 40 reads the start flag from the RAM, and ends the sub-process and proceeds to the sub-process of the “third evaluation and teaching process” of step S46 when the start flag is set to OFF (S531: NO).

On the other hand, when the start flag is set to ON (S531: YES), the control device 40 executes processing of steps S232 to S248 (see FIG. 15) among the sub-process of the “dual task process” executed by the control device 80 of the smartphone 5 as described above, and then ends the sub-process and proceeds to the sub-process of the “third evaluation and teaching process” of step S46. When the dual task flag is set to OFF in step S232 (S232: NO), the control device 40 ends the sub-process and proceeds to the sub-process of the “third evaluation and teaching process” of step S46.

Accordingly, the control device 40 voice-outputs the task via the speaker 105, and questions the walking user about the task. The control device 40 also receives the answer voice of the user to the task via the microphone 106, performs the voice recognition process on the answer voice, and analyzes the answered contents. For example, the control device 40 outputs the answer-correct sound such as “Pinpoon” via the speaker 105 to notify that the answer is correct. The control device 40 may also display a large white circle on the display 104 to notify that the answer is correct. For example, the control device 40 outputs the answer-incorrect sound such as “Boo-boo” via the speaker 105 to notify that the answer is incorrect. The control device 40 may also display a large X mark on the display 104 to notify that the answer is incorrect.

For example, a meal menu for one week of the user may be created and stored in advance in the EEPROM together with scheduled date and scheduled time of each meal. Then the control device 40 may create or select a question of the meal menu relative to a recent meal based on the stored meal menu for the one week, for example. The control device 40 may also create an answer to the question of the meal menu by analyzing the stored meal menu for the one week, and store the answer in the RAM.

Here, processing of step S233 executed by the control device 40 corresponds to the task creation unit 40C. Processing of step S234 executed by the control device 40 corresponds to the task output unit 40D. Processing of step S235 executed by the control device 40 corresponds to the voice recognition unit 40B. Processing of step S241 executed by the control device 40 corresponds to the determination unit 40E.

[Third Evaluation and Teaching Process]

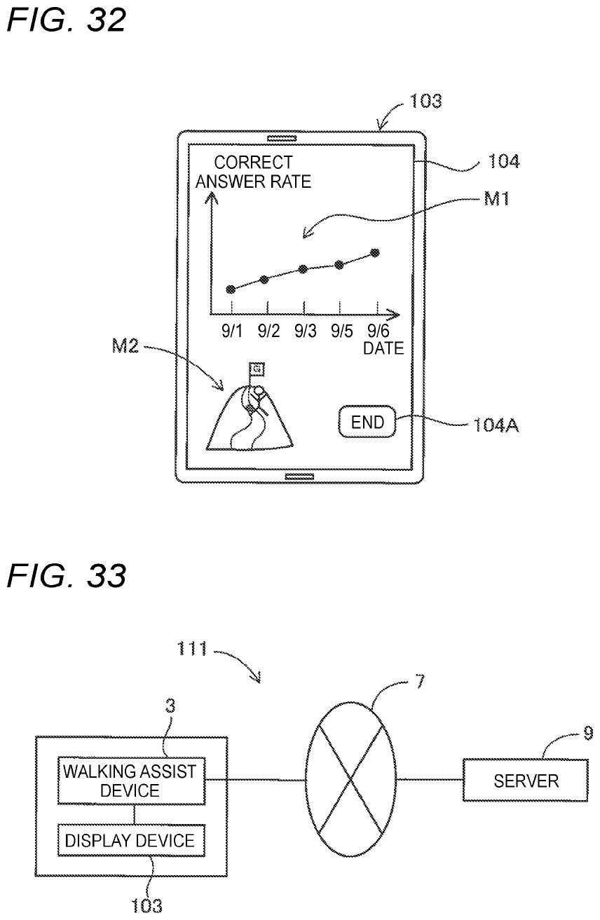

Next, the sub-process of the “third evaluation and teaching process” will be described with reference to FIGS. 31 and 32. As shown in FIG. 31, first, in step S541, the control device 40 reads the start flag from the RAM, and ends the sub-process and ends the third walking training process when the start flag is set to ON (S541: NO).

On the other hand, when the start flag is set to OFF (S541: YES), the control device 40 executes processing of steps S262 to S268 (see FIG. 16) among the sub-process of the “evaluation and teaching process” executed by the control device 80 of the smartphone 5 as described above, and then ends the sub-process and ends the third walking training process. When the answer flag is set to OFF in step S262 (S262: NO), the control device 40 ends the sub-process and ends the third walking training process.

In step S265, the control device 40 reads, from the EEPROM, the number of correct answers among the answers answered by the user to the tasks of several days in the past, the correct answer rate, the level of achievement relative to the target correct answer rate, and the like, creates a graph or design for such parameters, displays the graphed or designed parameters on the display 104 as the evaluation result or the reward, and then proceeds to step S266. Accordingly, processing of steps S263 to S265 executed by the control device 40 correspond to the information collection and evaluation unit 40F, the information providing unit 40G, and the teaching unit 40H.

For example, as shown in FIG. 32, the control device 40 reads the correct answer rate of the past five days from the EEPROM, creates the correct answer rate transition map M1, and displays the correct answer rate transition map M1 on the display 104 as the evaluation result. The control device 40 also reads the level of achievement relative to the current target correct answer rate from the EEPROM, creates the level-of-achievement map M2 in which the position of the user who climbs toward the mountain top goal indicates the level of achievement relative to the target correct answer rate, and displays the level-of-achievement map M2 on the display 104 as the reward for the user. The control device 40 also displays a display end button 104A on the display 104.

Here, the control device 40 of the walking assist device 3 functions as an example of a task load unit, an answer analysis unit, a notification unit, an analysis information collection unit, an answer evaluation unit, a teaching unit, a reward granting unit, an elapsed time measurement unit, and a time determination unit. The speaker 105 functions as an example of a voice output unit. The microphone 106 functions as an example of a voice input unit.

As described in detail above, in the walking support system for combined training 101 according to the third embodiment, the user presses the dual task training button 90D of the operation panel screen 90 displayed on the display 104 of the display device 103, and then presses the start button 90A. As a result, the user can answer by voice in consideration of the answer to the task each time the task is voice-output from the speaker 105 while performing the walking training during which the user walks while gripping the left and right grip handles 20L and 20R of the walking assist device 3. Accordingly, the user can perform the dual task training in which the walking training is performed at the same time while answering by voice in consideration of the answer to the task, and thus the walking training can be performed more effectively.

When the end button 90B of the operation panel screen 90 is pressed, the control device 40 displays (teaches) the evaluation information (for example, the correct answer rate transition map M1 indicating the change in the correct answer rate for each day or the level-of-achievement map M2) related to the execution degree of the task executed by the user on the display 104. As a result, the interest of the user in providing the voice answer to the task during the walking training can be effectively attracted by the evaluation information displayed on the display 104, and the motivation for the user to continuously perform the dual task training can be increased.

### Fourth Embodiment

Next, a walking support system for combined training 111 according to a fourth embodiment will be described with reference to FIGS. 33 to 36. The same reference numerals as those of the walking support system for combined training 91 according to the second embodiment denote the same or corresponding parts as those of the walking support system for combined training 91 according to the second embodiment. The same reference numerals as those of the walking support system for combined training 101 according to the third embodiment denote the same or corresponding parts as those of the walking support system for combined training 101 according to the third embodiment.

The walking support system for combined training 111 according to the fourth embodiment has substantially the same configuration as the walking support system for combined training 101 according to the third embodiment. However, as shown in FIG. 33, the walking support system for combined training 111 includes the walking assist device 3, which includes the display device 103, and the server 9. The walking assist device 3 is connected to the network 7, such as the Internet, via the communication device 17 (see FIG. 25). The server 9 is connected to the network 7, such as the Internet, via the communication device 97 (see FIG. 19). Accordingly, the walking assist device 3 and the server 9 are configured to be capable of transmitting and receiving information data to and from each other by wireless communication through Wi-Fi or the like via the network 7 such as the Internet.

[Details of Walking Training Control]

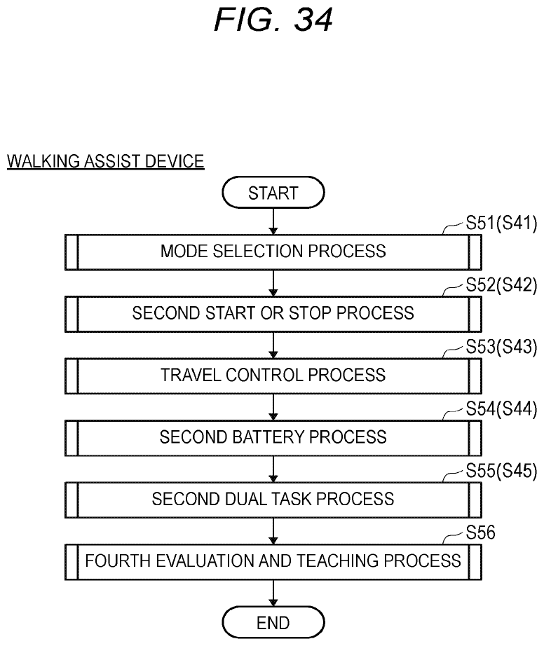

Next, a processing procedure performed by each of the control devices 40 and 93 (see FIGS. 19 and 25) when the user selects and performs one of the normal walking training and the dual task (double task) training through using the walking support system for combined training 111 configured as described above will be described with reference to FIGS. 34 to 36.

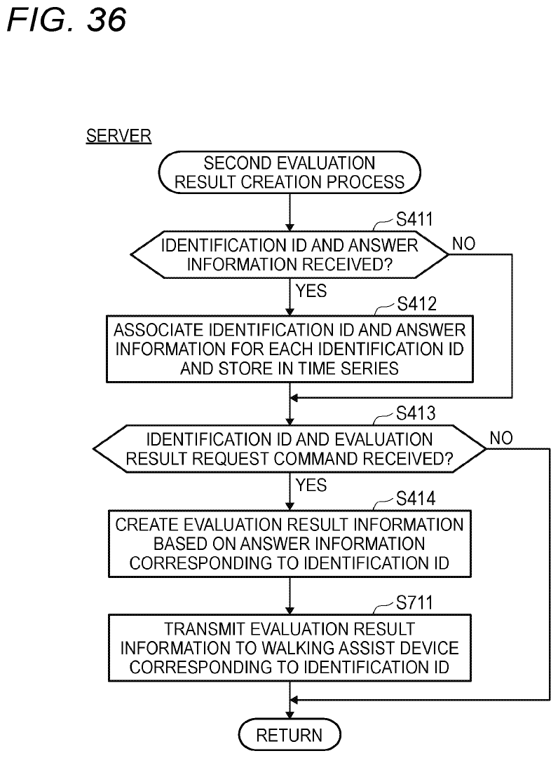

Specifically, when the user turns on the main switch 12, the control device 40 of the walking assist device 3 is activated to execute a “fourth walking training process” shown in FIG. 34 at predetermined time intervals (for example, intervals of several [ms]). The control device 40 displays the operation panel screen 90 (see FIG. 24) on the display 104. The control device 93 of the server 9 executes a “second evaluation result creation process” shown in FIG. 36 at predetermined time intervals (for example, intervals of several [ms]).

[Fourth Walking Training Process]

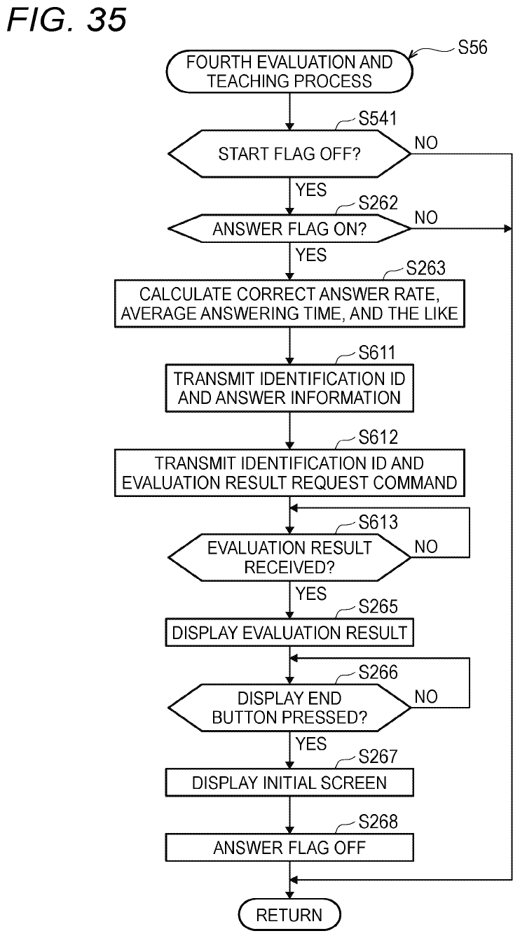

Next, the “fourth walking training process” executed by the control device 40 of the walking assist device 3 will be described with reference to FIGS. 34 and 35. Programs shown in flowcharts of FIGS. 34 and 35 are stored in advance in the EEPROM of the control device 40.

As shown in FIG. 34, in steps S51 to S55, the control device 40 executes steps S41 to S45 of the above-described “third walking training process” (see FIG. 27), and then executes the sub-process of the “fourth evaluation and teaching process” in step S56, and then ends the fourth walking training process.

[Fourth Evaluation and Teaching Process]

Next, the sub-process of the “fourth evaluation and teaching process” will be described with reference to FIG. 35. As shown in FIG. 35, the sub-process of the “fourth evaluation and teaching process” is substantially the same control process as the sub-process of the “third evaluation and teaching process” (see FIG. 31). However, the sub-process of the “fourth evaluation and teaching process” is different from the sub-process of the “third evaluation and teaching process” in that the control device 40 executes processing of steps S611 to S613 instead of processing of step S264 (see FIG. 31).

Specifically, in steps S611 to S613, the control device 40 executes processing of steps S311 to S313 (see FIG. 16) among the sub-process of the “second evaluation and teaching process” (see FIG. 21) executed by the control device 80 of the smartphone 5. However, in step S611, the control device 40 adds an identification ID for identifying the walking assist device 3 to the answer information and transmits the answer information to the server 9 via the communication device 17. The identification ID for identifying the walking assist device 3 is stored in the EEPROM in advance.

In step S612, the control device 40 adds the identification ID for identifying the walking assist device 3 to the evaluation result request command and transmits the evaluation result request command to the server 9 via the communication device 17. In step S613, the control device 40 receives the evaluation result information (evaluation information), where the identification ID for identifying the walking assist device 3 is added, from the server 9 via the communication device 17.

As a result, in step S265, the control device 40 can display, on the display 104, the evaluation result information such as the number of correct answers among the answers answered by the user to the tasks of several days in the past, the correct answer rate, and the level of achievement relative to the target correct answer rate. Accordingly, processing of steps S263 to S611 to S613 to S265 executed by the control device 40 corresponds to the information providing unit 40G and the teaching unit 40H (see FIG. 26).

[Second Evaluation Result Creation Process]

Next, the “second evaluation result creation process” executed by the control device 93 (see FIG. 19) of the server 9 will be described with reference to FIG. 36. As shown in FIG. 36, the sub-process of the “second evaluation result creation process” is substantially the same control process as the sub-process of the “evaluation result creation process” (see FIG. 22). However, the sub-process of the “second evaluation result creation process” is different from the sub-process of the “evaluation result creation process” in that the control device 93 executes processing of step S711 instead of step S415.

Specifically, in steps S411 to S414, the control device 93 associates the “answer information” received via the communication device 97 with the “identification ID” for identifying the walking assist device 3, and stores the “answer information” in the storage device 96 in time series for each identification ID together with the information on date and time. When the control device 93 receives the “evaluation result request command” and the “identification ID” for identifying the walking assist device 3 via the communication device 97, the control device 93 creates the “evaluation result information (evaluation information)” based on the “answer information” stored in association with the “identification ID”.

Then, in step S711, the control device 93 adds the “identification ID” of the walking assist device 3 received together with the “evaluation result request command” to the “evaluation result information”, transmits the “evaluation result information” to the walking assist device 3 corresponding to the “identification ID” via the communication device 97, and ends the sub-process. Accordingly, processing of steps S411 to S711 executed by the control device 93 functions as the information collection and evaluation unit 40F (see FIG. 26) of the control device 40 of the walking assist device 3.

Here, the communication device 17 functions as an example of a device communication device. The communication device 97 functions as an example of a server communication device. The control device 40 of the walking assist device 3 functions as an example of a task load unit, an answer analysis unit, a notification unit, an analysis information collection unit, a teaching unit, a reward granting unit, an elapsed time measurement unit, a time determination unit, a device transmission unit, and a device reception unit. The speaker 105 functions as an example of a voice output unit. The microphone 106 functions as an example of a voice input unit. The control device 93 of the server 9 functions as an example of a server reception unit, an answer evaluation unit, and a server transmission unit.

As described in detail above, in the walking support system for combined training 111 according to the fourth embodiment, the user presses the dual task training button 90D of the operation panel screen 90 displayed on the display 104 of the display device 103, and then presses the start button 90A. As a result, the user can answer by voice in consideration of the answer to the task each time the task is voice-output from the speaker 105 while performing the walking training during which the user walks while gripping the left and right grip handles 20L and 20R of the walking assist device 3. Accordingly, the user can perform the dual task training in which the walking training is performed at the same time while answering by voice in consideration of the answer to the task, and thus the walking training can be performed more effectively.

When the end button 90B (see FIG. 24) of the operation panel screen 90 is pressed, the control device 40 requests the server 9 for the “evaluation result information” of past several days as desired or of preset past several days. Then, based on the “evaluation result information” received from the server 9, the control device 40 creates the evaluation information (for example, the correct answer rate transition map M1 indicating the change in the correct answer rate for each day or the level-of-achievement map M2) related to the execution degree of the task executed by the user, and displays (teaches) the evaluation information on the display 104.

As a result, the interest of the user in providing the voice answer to the task during the walking training can be effectively attracted by the evaluation information displayed on the display 104, and the motivation for the user to continuously perform the dual task training can be increased. Moreover, since the server 9 creates the “evaluation result information” of the preset past several days, a processing load of the control device 40 of the walking assist device 3 can be reduced. Moreover, the server 9 can transmit the “evaluation result information” not only to the walking assist device 3 but also to other processing devices such as personal computers.

The present disclosure is not limited to the first to fourth embodiments, and it is needless to say that various improvements, modifications, additions, and deletions can be made without departing from the gist of the present disclosure. In the following description, the same reference numerals as those of configurations of the walking assist device 3, the smartphone 5, and the server 9 according to the first to fourth embodiments shown in FIGS. 1 to 36 denote the same or corresponding parts as those of the configurations of the walking assist device 3, the smartphone 5, and the server 9 according to the first to fourth embodiments.

### Another First Embodiment

(A) For example, in the sub-process of the “dual task process” of the “first walking training process” shown in FIG. 15, in step S233, the control device 80 may create a conversation to be spoken to another person such as “It's a nice weather today” as the task to be spoken to the user. Then, in step S241, the control device 80 may determine whether contents of a conversation obtained by analyzing contents of the answer voice of the user correspond to the conversation created in step S233.

Then the control device 80 may determine that the answer voice is a “correct answer” when the contents thereof correspond to the conversation created in step S233 and proceed to step S242. On the other hand, the control device 80 may determine that the answer voice is an “incorrect answer” when the contents thereof do not correspond to the conversation created in step S233 and proceed to step S244.

As a result, the user can reply by voice (answer) in consideration of the reply to a topic of the conversation each time the conversation is voice-output from the speaker 82 of the smartphone 5 while performing the walking training during which the user walks while gripping the left and right grip handles 20L and 20R of the walking assist device 3. Accordingly, the user can perform the dual task training in which the walking training is performed at the same time while replying by voice in consideration of the reply to the topic of the conversation, and thus the walking training can be performed more effectively.

### Another Second Embodiment

(B) For example, in the sub-process of the “dual task process” of the “first walking training process” shown in FIG. 15, in step S236, the control device 80 may acquire a moving speed of the walking assist device 3, that is, a walking speed of the user. When the walking speed of the user decreases or when the user stops, the contents of the task to be output via the speaker 82 in step S239 may be changed. As a result, even if the user is in trouble since the user does not know the answer to the task, the user can answer the task questioned again and resume walking.

### Another Third Embodiment

(C) For example, although the speaker 105 and the microphone 106 are provided in the display device 103 in the walking support system for combined training 101 according to the third embodiment, the speaker 105 and the microphone 106 may be provided at any position of the walking assist device 3 as desired. For example, the speaker 105 and the microphone 106 may be arranged on an upper end surface of the left handle cover 30L. As a result, a structure of the display device 103 can be simplified.

### Another Fourth Embodiment

(D) For example, in the walking assist device 3 according to the first embodiment, the travel drive devices 64L and 64R and the battery B may be removed while only the travel speed detection devices 64LE and 64RE that detect the rotation speeds of the rear wheels 60RL and 60RR are provided such that the walking assist device becomes a rollator walker that assists the walking of the user without electric assist. The control device 80 of the smartphone 5 may execute the first walking training process shown in FIG. 12. As a result, the user can perform the dual task training in which the walking training during which the user pushes the rollator walker and walks is performed at the same time while answering by voice in consideration of the answer to the task, and thus the walking training can be performed more effectively.

### Another Fifth Embodiment

(E) For example, in the walking assist device 3 according to the first embodiment, the grip handles 20L and 20R may face substantially upward, and the grip handles 20L and 20R may be movable in the front-rear direction. When the user walks while swinging arms in the state where the grip handles 20L and 20R are gripped, the walking assist device 3 may be configured to move in accordance with the arm-swinging walking of the user.

Then the control device 80 of the smartphone 5 may execute the first walking training process shown in FIG. 12 when the user performs the arm-swinging walking while gripping the grip handles 20L and 20R. As a result, the user can perform the walking training during which the arm-swinging walking is performed in the state where the grip handles 20L and 20R are gripped at the same time while answering by voice in consideration of the answer to the task. Accordingly, the user can perform both a body-using task, which is the arm-swinging walking, and a brain-using task, and thus an effect of the walking training can be further improved.

### Another Sixth Embodiment

(F) For example, in the walking assist device 3 according to the first embodiment, a hand grip or the like biased by a coil or a compression spring may be attached to each of the grip handles 20L and 20R so as to be opened in a substantially V shape in a side view such that grip strength of the user can be trained.

In the sub-process of the “dual task process” of the “first walking training process” shown in FIG. 15, the control device 80 of the smartphone 5 may alternately create an instruction of “Please grip the hand grip” and an instruction of “Please release the hand grip” at predetermined time intervals (for example, at intervals of about 15 seconds to 30 seconds) in steps S233 and S234, and may voice-output the instructions via the speaker 82 at the predetermined time intervals.

As a result, the user can perform the dual task training in which the walking training is performed at the same time while alternately performing exercises of gripping and releasing the hand grip or the like at the predetermined time intervals (for example, the intervals of about 15 seconds to 30 seconds), and thus the walking training can be performed more effectively.

The control device 80 of the smartphone 5 may also voice-output the calculation question, the four-basic-math-operation question, the question of the meal menu, and the like to the user via the speaker 82 during a period between a voice-output of an operation instruction of the hand grip or the like and a voice-output of a next operation instruction of the hand grip or the like. As a result, multi-task training, in which the walking training is performed at the same time while a body-using task and a brain-using task are both given to the user, is performed, and thus the walking training can be performed more effectively.

### Another Seventh Embodiment

(G) For example, although the level-of-achievement map M2 is displayed on the displays 81 and 104 to serve as the “reward” to the user in the first embodiment to the fourth embodiment, an economic “reward” such as a number of points or a number of coins given to the user may be displayed instead of the level-of-achievement map M2. As a result, the interest of the user in providing the voice answer to the task during the walking training can be effectively attracted, and the motivation for the user to continuously perform the dual task training can be increased.

What is claimed is:

1. A walking support system for a combined training comprising:
a walking assist device configured to move in accordance with an operation of a user to assist a walking of the user;
a voice output unit configured to output a voice; and
a task load unit configured to perform a voice-output for a task to the user via the voice output unit when the walking assist device is used by the user during the walking, the task being answerable by the voice.

2. The walking support system for the combined training according to claim 1, further comprising:
a voice input unit configured to receive a voice answer to the task from the user;
an answer analysis unit configured to analyze the voice answer received via the voice input unit; and
a notification unit configured to notify an analysis information of the voice answer provided by the answer analysis unit.

3. The walking support system for the combined training according to claim 2, further comprising:
an analysis information collection unit configured to collect the analysis information of the voice answer provided by the answer analysis unit;
an answer evaluation unit configured to evaluate an execution degree of the task based on the analysis information collected by the analysis information collection unit; and
a teaching unit configured to teach an evaluation information related to the execution degree of the task evaluated by the answer evaluation unit.

4. The walking support system for the combined training according to claim 3, further comprising:
a reward granting unit configured to determine and grant a reward to the user in accordance with the execution degree of the task evaluated by the answer evaluation unit.

5. The walking support system for the combined training according to claim 1, wherein the walking assist device includes a device communication device,
wherein the walking support system further comprises a mobile terminal including a terminal communication device which is communicable with the device communication device, and
wherein the mobile terminal includes the voice output unit and the task load unit.

6. The walking support system for the combined training according to claim 5, wherein the walking assist device includes an attachment member to which the mobile terminal is detachably attached.

7. The walking support system for the combined training according to claim 2, further comprising:
an elapsed time measurement unit configured to measure an elapsed time elapsed after the voice-output the task is performed via the task load unit; and
a time determination unit configured to determine whether the elapsed time reaches a predetermined first elapsed time in a state where the voice answer to the task is not received via the voice input unit,
wherein when the time determination unit determines that the elapsed time reaches the predetermined first elapsed time, the task load unit performs again the voice-output of the task via the voice output unit.

8. The walking support system for the combined training according to claim 2, further comprising:
an elapsed time measurement unit configured to measure an elapsed time elapsed after the voice-output of the task is performed via the task load unit; and
a time determination unit configured to determine whether the elapsed time reaches a predetermined first elapsed time in a state where the voice answer to the task is not received via the voice input unit,
wherein when the time determination unit determines that the elapsed time reaches the predetermined first elapsed time, the task load unit changes the task to another task and performs the voice-output of the another task via the voice output unit.

9. The walking support system for the combined training according to claim 3, wherein the walking assist device includes a device communication device,
wherein the walking support system further comprises: a mobile terminal including a terminal communication device which is communicable with the device communication device; and
a server including a server communication device which is communicable with the terminal communication device,
wherein the mobile terminal includes:
the voice output unit, the task load unit, the voice input unit, the answer analysis unit, the notification unit, the analysis information collection unit, and the teaching unit;
a terminal transmission unit configured to transmit the analysis information collected by the analysis information collection unit to the server via the terminal communication device; and
a terminal reception unit configured to receive the evaluation information related to the execution degree of the task from the server via the terminal communication device, and
wherein the server includes:
a server reception unit configured to receive the analysis information collected by the analysis information collection unit via the server communication device;
the answer evaluation unit configured to evaluate the execution degree of the task based on the analysis information which is received by the server reception unit and collected by the analysis information collection unit; and
a server transmission unit configured to transmit the evaluation information related to the execution degree of the task evaluated by the answer evaluation unit to the mobile terminal via the server communication device.

10. The walking support system for the combined training according to claim 3, wherein the walking assist device includes a device communication device,
wherein the walking support system further comprises a server including a server communication device which is communicable with the device communication device,
wherein the walking assist device includes:
the voice output unit, the task load unit, the voice input unit, the answer analysis unit, the notification unit, the analysis information collection unit, and the teaching unit;
a device transmission unit configured to transmit the analysis information collected by the analysis information collection unit to the server via the device communication device; and
a device reception unit configured to receive the evaluation information related to the execution degree of the task from the server via the device communication device, and
the server includes:
a server reception unit configured to receive the analysis information collected by the analysis information collection unit via the server communication device;
the answer evaluation unit configured to evaluate the execution degree of the task based on the analysis information which is received by the server reception unit and collected by the analysis information collection unit; and
a server transmission unit configured to transmit the evaluation information related to the execution degree of the task evaluated by the answer evaluation unit to the walking assist device via the server communication device.

11. The walking support system for the combined training according to claim 1, wherein the task includes at least one of a calculation question, a four-basic-math-operation question, a quiz question, and a question of a menu of a meal eaten by the user.
